SEQUENCE LISTING (15 sequences)

SEQ ID NO: 1 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 2 - protein, 5 aa, Artificial Sequence
  CDR
SYYMH

SEQ ID NO: 3 - protein, 17 aa, Artificial Sequence
  CDR
EISPFGGRTNYNEKFKS

SEQ ID NO: 4 - protein, 9 aa, Artificial Sequence
  CDR
ERPLYASDL

SEQ ID NO: 5 - protein, 11 aa, Artificial Sequence
  CDR
RASQGISSALA

SEQ ID NO: 6 - protein, 7 aa, Artificial Sequence
  CDR
SASYRYT

SEQ ID NO: 7 - protein, 9 aa, Artificial Sequence
  CDR
QQRYSLWRT

SEQ ID NO: 8 - protein, 444 aa, Artificial Sequence
  humanized antibody heavy chain sequence
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 214 aa, Artificial Sequence
  full length light chain
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 443 aa, Artificial Sequence
  full length heavy chain without C-terminal lysine
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 11 - protein, 118 aa, Artificial Sequence
  heavy chain variable region
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 12 - protein, 107 aa, Artificial Sequence
  light chain variable region
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 13 - protein, 7 aa, Artificial Sequence
  CDR
GYTFTSY

SEQ ID NO: 14 - protein, 10 aa, Artificial Sequence
  CDR
GYTFTSYYMH

SEQ ID NO: 15 - protein, 7 aa, Artificial Sequence
  CDR
ISPFGGR

(19) United States
(12) Patent Application Publication
(10) Pub. No.: US 2016/0152734 A1
(43) Pub. Date: Jun. 2, 2016

(54) TREATMENT WITH ANTI-PCSK9 ANTIBODIES

(71) Applicant: PFIZER INC., New York, NY (US)

(72) Inventor: Chandrasekhar UDATA, San Diego, CA (US)

(73) Assignee: PFIZER INC., NEW YORK, NY (US)

(21) Appl. No.: 15/016,873

(22) Filed: Feb. 5, 2016

Related U.S. Application Data
(62) Division of application No. 14/232,559, filed on Jan. 13, 2014.
(60) Provisional application No. 61/507,865, filed on Jul. 14, 2011.
(60) Provisional application No. 61/614,312, filed on Mar. 22, 2012.
(60) Provisional application No. 61/643,063, filed on May 4, 2012.

(51) Int. Cl. C07K 16/40; A61K 9/00

(52) U.S. Cl. CPC C07K 16/40; A61K 9/0019; C07K 2317/76; A61K 2039/545

(57) ABSTRACT

The present invention concerns dosages for the treatment of human patients susceptible to or diagnosed with a disorder characterized by marked elevations of low density lipoprotein particles in the plasma with a PCSK9 antagonist antiboyd alone or in combination with a statin.

### RELATED APPLICATIONS

This application claims the benefits of U.S. Provisional Application No. 61/507,865 filed Jul. 14, 2011, U.S. Provisional Application No. 61/614,312 filed Mar. 22, 2012, and U.S. Provisional Application No. 61/643,063 filed May 4, 2012, all of which are hereby incorporated by reference in their entireties.

### FIELD

The present invention concerns therapeutic regimens for treatment of disorders characterized by marked elevations of low density lipoprotein (“LDL”) particles in the plasma. The subject therapeutic regimens involve administration of an anti-proprotein convertase subtilisin kexin type 9 (PCSK9) antibody, alone or in combination with a statin. The subject therapeutic regimens provide for enhanced reduction of LDL-cholesterol levels in blood, and can be used in the prevention and/or treatment of cholesterol and lipoprotein metabolism disorders, including familial hypercholesterolemia, atherogenic dyslipidemia, atherosclerosis, acute coronary syndrome and, more generally, cardiovascular disease.

### BACKGROUND

Millions of people in the U.S. are at risk for heart disease and resulting cardiac events. Cardiovascular disease and underlying atherosclerosis is the leading cause of death among all demographic groups, despite the availability of therapies directed at its multiple risk factors. Atherosclerosis is a disease of the arteries and is responsible for coronary heart disease associated with many deaths in industrialized countries. Several risk factors for coronary heart disease have now been identified: dyslipidemias, hypertension, diabetes, smoking, poor diet, inactivity and stress. The most clinically relevant and common dyslipidemias are characterized by an increase in beta-lipoproteins (very low density lipoprotein (VLDL) and LDL) with hypercholesterolemia in the absence or presence of hypertriglyceridemia. Fredrickson et al., 1967, N Engl J Med. 276:34-42, 94-103, 148-156, 215-225, and 273-281. There is a long-felt significant unmet need with respect to cardiovascular disease with 60-70% of cardiovascular events, heart attacks and strokes occurring despite the treatment with statins (the current standard of care in atherosclerosis). Moreover, new guidelines suggest that even lower LDL levels should be achieved in order to protect high risk patients from premature cardiovascular disease (National Cholesterol Education Program (NCEP) 2004).

PCSK9 has been implicated as a major regulator of plasma low density lipoprotein cholesterol (LDL-C) and has emerged as a promising target for prevention and treatment of coronary heart disease (CHD). Hooper et al., 2011, Expert Opin Ther Targets 15(2):157-68. Human genetic studies identified gain-of-function mutations, which were associated with elevated serum levels of LDL-C and premature and incidences of CHD, whereas loss-of-function mutations were associated with low LDL-C and reduced risk of CHD. Abifadel, 2003, Nat Genet. 43(2):154-6; Cohen, 2005, Nat Genet. 37(2):161-5; Cohen, 2006, N Engl J Med. 354(12):1264-72; Kotowski, 2006, Am J Hum Genet. 78(3):410-22. In humans, the complete loss of PCSK9 results in low serum LDL-C of <20 mg/dL, in otherwise healthy subjects. Hooper, 2007, 193(2):445-8; Zhao, 2006, Am J Hum Genet. 79(3):514-523.

PCSK9 belongs to the subtilisin family of serine proteases and is formed by an N-terminal prodomain, a subtilisin-like catalytic domain and a C-terminal cysteine/histidine-rich domain (CHRD). Highly expressed in the liver, PCSK9 is secreted after the autocatalytic cleavage of the prodomain, which remains non-covalently associated with the catalytic domain. The catalytic domain of PCSK9 binds to the epidermal growth factor-like repeat A (EGF-A) domain of low density lipoprotein receptor (LDLR) at serum pH of 7.4 and higher affinity at endosomes pH of approximately 5.5-6.0. Bottomley, 2009, J Biol Chem. 284(2):1313-23. The C-terminal domain is involved in the internalization of the LDLR-PCSK9 complex, while not binding to catalytic domain. Nassoury, 2007, Traffic 8(7):950; Ni, 2010, J Biol Chem. 285(17):12882-91; Zhang, 2008, Proc Natl Acad Sci USA, 2008, 105(35):13045-50. Both functionalities of PCSK9 are required for targeting the LDLR-PCSK9 complex for lysosomal degradation and lowering LDL-C, which is in agreement with mutations in both domains linked to loss-of-function and gain-of-function. Lambert, 2009, Atherosclerosis 203(1):1-7.

Various therapeutic approaches for inhibiting PCSK9 are currently in development, including gene silencing by siRNA or anti-sense oligonucleotides and disruption of the PCSK9-LDLR interaction by antibodies. Brautbar et al., 2011, Nature Reviews Cardiology 8, 253-265. For example, Chan, 2009, and Ni, 2011, each report an anti-PCSK9 monoclonal antibody having LDL-C lowering activity in mice and non-human primates; the half-life of each antibody was reported as approximately 61 h and 77 h, respectively, in non-human primates when administered at 3 mg/kg of the PCSK9 antagonist antibody. Chan, 2009, Proc Natl Acad Sci USA 106(24):9820-5; Ni, 2011, J Lipid Res. 52(1):78-86. The PSCK9 antagonist antibody 7D4 has been reported to effectively reduce serum cholesterol levels in cynomoglus monkey; the half-life of 7D4 in cynomolgus monkeys was less than 2 days at a single dose of 10 mg/kg of the PCSK9 antagonist antibody. PCT Patent Application Publication No. WO 2010/029513.

From the information available in the art, and prior to the present invention, it remained unclear whether low, infrequent doses of PCSK9 antagonist antibody would be effective to reduce hypercholesterolemia and the associated incidence of CVD in human patients and, if so, what dosage regimens are needed for such in vivo effectiveness.

### SUMMARY

This invention relates to therapeutic regimens for prolonged reduction of LDL-C levels in blood by inhibiting PCSK9 activity and the corresponding effects of PCSK9 on LDL-C plasma levels.

In some embodiments, the invention provides a method for the treatment of a human patient susceptible to or diagnosed with a disorder characterized by an elevated low-density lipoprotein cholesterol (LDL-C) level in the blood, the method comprising administering to the patient an initial dose of at least about 0.25 mg/kg, 0.5 mg/kg, 1 mg/kg, 1.5 mg/kg, 2 mg/kg, 3 mg/kg, 4 mg/kg, 5 mg/kg, 6 mg/kg, 8 mg/kg, 12 mg/kg, 50 mg, 100 mg, 150 mg, 200 mg, 250 mg, 300 mg, 350 mg, or 400 mg of a proprotein convertase subtilisin kexin type 9 (PCSK9) antagonist antibody; and administering to the patient a plurality of subsequent doses of the antibody in an amount that is about the same or less than the initial dose, wherein the initial dose and the first subsequent and additional subsequent doses are separated in time from each other by at least about one, two, three, or four weeks. The invention can be practiced using, for example, the PCSK9 antagonist antibody L1L3. In some embodiments, the invention can be practiced using an antibody comprising three CDRS from a heavy chain variable region having the amino acid sequence shown in SEQ ID NO: 11 and three CDRS from a light chain variable region having the amino acid sequence shown in SEQ ID NO: 12.

In some embodiments, the initial dose can be about 0.25 mg/kg, about 0.5 mg/kg, about 1 mg/kg or about 1.5 mg/kg, and the initial dose and the first subsequent dose and additional subsequent doses can be separated from each other in time by about one week.

In other embodiments, the initial dose can be about 2 mg/kg, about 4 mg/kg, about 8 mg/kg, or about 12 mg/kg, and the initial dose and the first subsequent dose and additional subsequent doses can be separated from each other in time by at least about two weeks.

In other embodiments, the initial dose can be about 50 mg, about 100 mg, about 150 mg, or about 175 mg, and the initial dose and the first subsequent dose and additional subsequent doses can be separated from each other in time by at least about two weeks.

In other embodiments, the initial dose can be about 3 mg/kg or about 6 mg/kg, and the initial dose and the first subsequent dose and additional subsequent doses can be separated from each other in time by at least about four weeks. In other embodiments, the initial dose can be about 200 mg or about 300 mg, and the initial dose and the first subsequent dose and additional subsequent doses can be separated from each other in time by at least about four weeks. In some embodiments, the PCSK9 antagonist antibody is administered subcutaneously. In some embodiments, the PCSK9 antagonist antibody is administered intravenously.

In some embodiments, the initial dose and the first subsequent dose and additional subsequent doses can be separated from each other in time by about four weeks. In some embodiments, the initial dose and the first subsequent dose and additional subsequent doses can be separated from each other in time by about eight weeks. Each of the plurality of subsequent doses can be about the same amount or less than the initial dose.

In some embodiments, the disorder can be hypercholesterolemia, dyslipidemia, atherosclerosis, cardiovascular disease, coronary heart disease, or acute coronary syndrome (ACS). The human patient may have a fasting total cholesterol level of, for example, about 600 mg/dL or greater prior to administration of the initial dose of PCSK9 antagonist antibody. The human patient may have a fasting LDL cholesterol level of, for example, about 130 mg/dL or greater prior to administration of the initial dose of PCSK9 antagonist antibody. In some embodiments, the human patient may have a fasting LDL cholesterol level of about 145 mg/dL or greater prior to administration of the initial dose of PCSK9 antagonist antibody.

In some embodiments, the patient is being treated with a statin. In some embodiments, the patient is being treated with a daily dose of a statin. In some embodiments, the human patient may have been administered an effective amount of a statin prior to administration of the initial dose of PCSK9 antagonist antibody. In some embodiments, the patient is on stable doses of a statin prior to administration of an initial dose of PCSK9 antibody. The stable doses can be, for example, a daily dose or an every-other-day dose. In some embodiments, the human patient is on a daily stable dose of a statin for at least about two, three, four, five, or six weeks prior to administration of the initial dose of PCSK9 antagonist antibody. In some embodiments, the human patient on stable doses of a statin has a fasting LDL cholesterol level of, for example, about 70 or 80 mg/dL or greater prior to administration of the initial dose of PCSK9 antagonist antibody.

In some embodiments, the method further comprises administering an effective amount of a statin.

In some embodiments, the initial dose of PCSK9 antagonist antibody can be about 3 mg/kg or about 6 mg/kg, and the initial dose and the first subsequent dose and additional subsequent doses can be separated from each other in time by about four weeks or about one month. In some embodiments, the initial dose of PCSK9 antagonist antibody can be about 200 mg or about 300 mg, and the initial dose and the first subsequent dose and additional subsequent doses can be separated from each other in time by about four weeks or about one month.

The statin can be, for example, atorvastatin, cerivastatin, fluvastatin, lovastatin, mevastatin, pitavastatin, pravastatin, rosuvastatin, simvastatin, or a combination therapy selected from the group consisting of simvastatin plus ezetimibe, lovastatin plus niacin, atorvastin plus amlodipine, and simvastatin plus niacin. In some embodiments, the statin dose can be, for example, 40 mg atorvastatin, 80 mg atorvastatin, 20 mg rosuvastatin, 40 mg rosuvastatin, 40 mg simvastatin, or 80 mg simvastatin. In some embodiments, the method comprises administering to the patient an initial dose of at least about 3 mg/kg or about 6 mg/kg of PCSK9 antagonist antibody L1L3; and administering to the patient a plurality of subsequent doses of the antibody in an amount that is about the same or less than the initial dose, wherein the initial dose and the first subsequent and additional subsequent doses are separated in time from each other by at least about four weeks, wherein the patient is being treated with a stable daily dose of a statin. In some embodiments, the stable daily dose of a statin can be 40 mg atorvastatin, 80 mg atorvastatin, 20 mg rosuvastatin, 40 mg rosuvastatin, 40 mg simvastatin, or 80 mg simvastatin.

In some embodiments, the method comprises administering to the patient an initial dose of at least about 200 mg or about 300 mg of PCSK9 antagonist antibody L1L3; and administering to the patient a plurality of subsequent doses of the antibody in an amount that is about the same or less than the initial dose, wherein the initial dose and the first subsequent and additional subsequent doses are separated in time from each other by at least about four weeks, wherein the patient is being treated with a stable daily dose of a statin. In some embodiments, the method comprises administering to the patient an initial dose of at least about 50 mg, about 100 mg, about 150 mg, or about 175 mg of PCSK9 antagonist antibody L1L3; and administering to the patient a plurality of subsequent doses of the antibody in an amount that is about the same or less than the initial dose, wherein the initial dose and the first subsequent and additional subsequent doses are separated in time from each other by at least about two weeks, wherein the patient is being treated with a stable daily dose of a statin. In some embodiments, the stable daily dose of a statin can be 40 mg atorvastatin, 80 mg atorvastatin, 20 mg rosuvastatin, 40 mg rosuvastatin, 40 mg simvastatin, or 80 mg simvastatin.

In some embodiments, the PCSK9 antagonist antibody is administered subcutaneously or intravenously.

The invention also provides article of manufacture, comprising a container, a composition within the container comprising a PCSK9 antagonist antibody, and a package insert containing instructions to administer an initial dose of PCSK9 antagonist antibody of at least about 0.25 mg/kg, 0.5 mg/kg, 1 mg/kg, 1.5 mg/kg, 2 mg/kg, 3 mg/kg, 4 mg/kg, 6 mg/kg, 8 mg/kg, 12 mg/kg, 50 mg, 100 mg, 150 mg, 200 mg, 250 mg, 300 mg, 350 mg or 400 mg, and at least one subsequent dose that is the same amount or less than the initial dose. In some embodiments, the invention can be practiced using an antibody comprising three CDRS from a heavy chain variable region having the amino acid sequence shown in SEQ ID NO: 11 and three CDRS from a light chain variable region having the amino acid sequence shown in SEQ ID NO: 12. In some embodiments, the invention can be practiced using the PCSK9 antagonist antibody L1L3.

The administration of the initial dose and subsequent doses can be separated in time by, for example, at least about one, at least about two, three, four, five, six, seven or eight weeks. In some embodiments, instructions can be, for example, for administration of an initial dose by intravenous injection and at least one subsequent dose by intravenous or subcutaneous injection. In other embodiments, instructions can be, for example, for administration of an initial dose by subcutaneous injection and at least one subsequent dose by intravenous or subcutaneous injection.

In some embodiments, a plurality of subsequent doses can be administered. The plurality of subsequent doses can be separated in time from each other by, for example, at least two, three, four, five, six, seven or eight weeks.

In some embodiments, the package insert can further include instructions for administration of the PCSK9 antagonist antibody to a patient being treated with a statin. The statin can be, for example, atorvastatin, cerivastatin, fluvastatin, lovastatin, mevastatin, pitavastatin, pravastatin, rosuvastatin, simvastatin, or a combination therapy selected from the group consisting of simvastatin plus ezetimibe, lovastatin plus niacin, atorvastin plus amlodipine, and simvastatin plus niacin.

In some embodiments, the article of manufacture can further comprise a label on or associated with the container that indicates that the composition can be used for treating a condition characterized by an elevated low-density lipoprotein cholesterol level in the blood. The label can indicate that the composition can be used for the treatment of, for example, hypercholesterolemia, atherogenic dyslipidemia, atherosclerosis, cardiovascular disease, and/or acute coronary syndrome (ACS).

### BRIEF DESCRIPTION OF THE DRAWINGS

FIG. 1 depicts a graph showing absolute fasting LDL-C levels in mg/dL after L1L3 antibody administration.

FIG. 2 depicts a graph showing the percentage change from baseline of fasting LDL-C levels after L1L3 antibody administration.

FIG. 3 depicts a graph showing the percentage change from baseline of fasting total cholesterol levels after L1L3 antibody administration.

FIG. 4 depicts a graph showing the percentage change from baseline of fasting apolipoprotein B levels after L1L3 antibody administration.

FIG. 5 depicts a graph showing the percentage change from baseline of fasting high density lipoprotein cholesterol levels after L1L3 antibody administration.

FIG. 6 depicts a graph showing the percentage change from baseline of fasting triglyceride lipoprotein cholesterol levels after L1L3 antibody administration.

FIG. 7A depicts a graph showing absolute fasting LDL-C levels in mg/dL after L1L3 antibody administration. FIG. 7B depicts a graph showing the percentage change from baseline of fasting LDL-C levels in mg/dL after L1L3 antibody administration.

FIG. 8 depicts a graph showing the percentage change from baseline of fasting LDL-C levels after L1L3 antibody administration, with or without statin present. X-axis indicates the dose amount of L1L3 in mg/kg of the PCSK9 antagonist antibody.

FIGS. 9A-F depicts simulated time profiles for L1L3 (A-C) and LDL-C (E-F). (A) and (D): L1L3 at 2 mg/kg of the PCSK9 antagonist antibody. (B) and (E): L1L3 at 6 mg/kg of the PCSK9 antagonist antibody. (C) and (F): Placebo. X-axis indicates time in days.

FIG. 10 depicts simulated time profiles for LDL-C after dosing with the indicated L1L3 dose amounts.

FIG. 11 depicts a schematic of the study design for L1L3 monotherapy.

FIG. 12 depicts a graph showing absolute fasting LDL-C levels in mg/dL after L1L3 antibody administration.

FIG. 13 depicts a graph showing the percentage change from baseline of fasting LDL-C levels after L1L3 antibody administration.

FIG. 14 depicts a table showing the mean percentage change from baseline of fasting LDL-C levels after L1L3 antibody administration.

FIG. 15 depicts a graph showing the percent change from baseline of fasting LDL-C levels after L1L3 antibody administration.

FIG. 16 depicts a graph showing the percent change from baseline of fasting LDL-C levels after L1L3 antibody administration, excluding subjects with missed doses.

### DETAILED DESCRIPTION

Provided herein are therapeutic regimens for treatment of disorders characterized by marked elevations of LDL particles in the plasma. The subject therapeutic regimens involve administration of a PCSK9 antagonist antibody, alone or in combination with a statin. The subject therapeutic regimens provide for prolonged reduction of LDL-cholesterol levels in blood, and can be used in the prevention and/or treatment of cholesterol and lipoprotein metabolism disorders, including familial hypercholesterolemia, atherogenic dyslipidemia, atherosclerosis, acute coronary syndrome (ACS), and, more generally, cardiovascular disease.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also any antigen binding fragment (i.e., “antigen-binding portion”) or single chain thereof, fusion proteins comprising an antibody, and any other modified configuration of the immunoglobulin molecule that comprises an antigen recognition site including, for example without limitation, scFv, single domain antibodies (e.g., shark and camelid antibodies), maxibodies, minibodies, intrabodies, diabodies, triabodies, tetrabodies, v-NAR and bis-scFv (see, e.g., Hollinger and Hudson, 2005, Nature Biotechnology 23(9): 1126-1136). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody, as used herein, refers to one or more fragments of an intact antibody that retain the ability to specifically bind to a given antigen (e.g., PCSK9). Antigen binding functions of an antibody can be performed by fragments of an intact antibody. Examples of binding fragments encompassed within the term “antigen binding portion” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., 1989, Nature 341:544-546), and an isolated complementarity determining region (CDR).

The term “monoclonal antibody” (Mab) refers to an antibody that is derived from a single copy or clone, including e.g., any eukaryotic, prokaryotic, or phage clone, and not the method by which it is produced. Preferably, a monoclonal antibody of the invention exists in a homogeneous or substantially homogeneous population.

“Humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity.

As used herein, “human antibody” means an antibody having an amino acid sequence corresponding to that of an antibody that can be produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al. Monoclonal Antibodies and Cancer Therapy, Alan R. Liss, p. 77, 1985; Boerner et al., 1991, J. Immunol., 147 (1):86-95; and U.S. Pat. No. 5,750,373.

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987). When choosing FR to flank subject CDRs, e.g., when humanizing or optimizing an antibody, FRs from antibodies which contain CDR1 and CDR2 sequences in the same canonical class are preferred.

A “CDR” of a variable domain are amino acid residues within the variable region that are identified in accordance with the definitions of the Kabat, Chothia, the acccumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., 1989, Nature 342:877-883. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., 1996, J. Mol. Biol., 262:732-745. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art a “constant region” of an antibody refers to the constant region of the antibody light chain or the constant region of the antibody heavy chain, either alone or in combination.

As used herein, the term “PCSK9” refers to any form of PCSK9 and variants thereof that retain at least part of the activity of PCSK9. Unless indicated differently, such as by specific reference to human PCSK9, PCSK9 includes all mammalian species of native sequence PCSK9, e.g., human, canine, feline, equine, and bovine. One exemplary human PCSK9 is found as Uniprot Accession Number Q8NBP7 (SEQ ID NO: 1).

As used herein, a “PCSK9 antagonist antibody” refers to an anti-PCSK9 antibody that is able to inhibit PCSK9 biological activity and/or downstream pathway(s) mediated by PCSK9 signaling, including PCSK9-mediated down-regulation of the LDLR, and PCSK9-mediated decrease in LDL blood clearance. A PCSK9 antagonist antibody encompasses antibodies that block, antagonize, suppress or reduce (to any degree including significantly) PCSK9 biological activity, including downstream pathways mediated by PCSK9 signaling, such as LDLR interaction and/or elicitation of a cellular response to PCSK9. For purpose of the present invention, it will be explicitly understood that the term “PCSK9 antagonist antibody” encompasses all the previously identified terms, titles, and functional states and characteristics whereby the PCSK9 itself, a PCSK9 biological activity (including but not limited to its ability to mediate any aspect of interaction with the LDLR, down regulation of LDLR, and decreased blood LDL clearance), or the consequences of the biological activity, are substantially nullified, decreased, or neutralized in any meaningful degree. In some embodiments, a PCSK9 antagonist antibody binds PCSK9 and prevents interaction with the LDLR. Examples of PCSK9 antagonist antibodies are provided in, e.g., U.S. Patent Application Publication No. 20100068199, which is herein incorporated by reference in its entirety.

As used herein a “full antagonist” is an antagonist which, at an effective concentration, essentially completely blocks a measurable effect of PCSK9. By a partial antagonist is meant an antagonist that is capable of partially blocking a measurable effect, but that, even at a highest concentration is not a full antagonist. By essentially completely is meant at least about 80%, preferably, at least about 90%, more preferably, at least about 95%, and most preferably, at least about 98% or 99% of the measurable effect is blocked. The relevant “measurable effects” are described herein and include down regulation of LDLR by a PCSK9 antagonist as assayed in Huh7 cells in vitro, in vivo decrease in blood (or plasma) levels of total cholesterol, and in vivo decrease in LDL levels in blood (or plasma).

As used herein, the term “clinically meaningful” means at least a 15% reduction in blood LDL-cholesterol levels in humans or at least a 15% reduction in total blood cholesterol in mice. It is clear that measurements in plasma or serum can serve as surrogates for measurement of levels in blood. As used herein, the term “dosing regimen” refers to the total course of treatment administered to a patient, e.g., treatment with a PCSK9 antagonist antibody.

As used herein, the term “continuous” in the context of the time in which the mean level of LDL cholesterol in blood is within a specific range of levels, means that the time the mean level is in that specific range is not interrupted by any time in which that mean level is not within that specific range of levels.

As used herein, the term “not continuous” in the context of the time in which the mean level of LDL cholesterol in blood is within a specific range of levels, means that the time the mean level is in that specific range is interrupted by some amount of time (e.g., 15 minutes, 20 minutes, 30 minutes, 45 minutes, 1 hour, 2 hours, 3 hours, 4, hours, 5 hours, 6 hours, 8 hours, 10 hours, 12 hours, 14 hours, 16 hours 18 hours, 20 hours, 24 hours 28 hours, 32 hours, 36 hours, 40 hours, 44 hours, 48 hours, 60 hours, 72 hours, 84 hours, 90 hours, or any range of time of having upper and lower limits of any of above the specifically stated times), in which that mean level is not within that specific range of levels.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, an antibody “interacts with” PCSK9 when the equilibrium dissociation constant is equal to or less than 20 nM, preferably less than about 6 nM, more preferably less than about 1 nM, most preferably less than about 0.2 nM, as measured by the methods disclosed in Example 2 of U.S. Patent Application Publication No. 20100068199.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PCSK9 epitope is an antibody that binds this epitope with greater affinity, avidity, more readily, and/or with greater duration than it binds to other PCSK9 epitopes or non-PCSK9 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976 J. Immunol., 117:587; and Kim et al., 1994, J. Immunol., 24:249).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

By an antibody with an epitope that “overlaps” with another (second) epitope or with a surface on PCSK9 that interacts with the EGF-like domain of the LDLR is meant the sharing of space in terms of the PCSK9 residues that are interacted with. To calculate the percent of overlap, for example, the percent overlap of the claimed antibody's PCSK9 epitope with the surface of PCSK9 which interacts with the EGF-like domain of the LDLR, the surface area of PCSK9 buried when in complex with the LDLR is calculated on a per-residue basis. The buried area is also calculated for these residues in the PCSK9:antibody complex. To prevent more than 100% possible overlap, surface area for residues that have higher buried surface area in the PCSK9:antibody complex than in LDLR:PCSK9 complex is set to values from the LDLR:PCSK9 complex (100%). Percent surface overlap is calculated by summing over all of the LDLR:PCSK9 interacting residues and is weighted by the interaction area.

A “functional Fc region” possesses at least one effector function of a native sequence Fc region. Exemplary “effector functions” include C1q binding; complement dependent cytotoxicity; Fc receptor binding; antibody-dependent cell-mediated cytotoxicity; phagocytosis; down-regulation of cell surface receptors (e.g., B cell receptor), etc. Such effector functions generally require the Fc region to be combined with a binding domain (e.g., an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g., from about one to about ten amino acid substitutions, and preferably, from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably, at least about 90% sequence identity therewith, more preferably, at least about 95%, at least about 96%, at least about 97%, at least about 98%, at least about 99% sequence identity therewith.

As used herein, the terms “atorvastatin”, “cerivastatin”, “fluvastatin”, “lovastatin”, “mevastatin”, “pitavastatin”, “pravastatin”, “rosuvastatin” and “simvastatin” include atorvastatin, cerivastatin, fluvastatin, lovastatin, mevastatin, pitavastatin, pravastatin, rosuvastatin, simvastatin, respectively, and any pharmaceutically acceptable salts, or stereoisomers, thereof. As used herein, the term “pharmaceutically acceptable salt” includes salts that are physiologically tolerated by a patient. Such salts are typically prepared from inorganic acids or bases and/or organic acids or bases. Examples of these acids and bases are well known to those of ordinary skill in the art.

As used herein, “treatment” is an approach for obtaining beneficial or desired clinical results. For purposes of this invention, beneficial or desired clinical results include, but are not limited to, one or more of the following: enhancement of LDL clearance and reducing incidence or amelioration of aberrant cholesterol and/or lipoprotein levels resulting from metabolic and/or eating disorders, or including familial hypercholesterolemia, atherogenic dyslipidemia, atherosclerosis, ACS, and, more generally, cardiovascular disease (CVD).

“Reducing incidence” means any of reducing severity (which can include reducing need for and/or amount of (e.g., exposure to) other drugs and/or therapies generally used for this condition. As is understood by those skilled in the art, individuals may vary in terms of their response to treatment, and, as such, for example, a “method of reducing incidence” reflects administering the PCSK9 antagonist antibody based on a reasonable expectation that such administration may likely cause such a reduction in incidence in that particular individual.

“Ameliorating” means a lessening or improvement of one or more symptoms as compared to not administering a PCSK9 antagonist antibody. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing hypercholesterolemia or one or more symptoms of dyslipidemia, atherosclerosis, cardiovascular disease, or coronary heart disease, decreasing the dose of other medications required to treat the disease, enhancing the effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual” or a “subject” is a mammal, more preferably, a human. Mammals also include, but are not limited to, farm animals, sport animals, pets, primates, horses, dogs, cats, mice and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy, 20th Ed., Mack Publishing, 2000).

The term “kₒₙ”, as used herein, refers to the rate constant for association of an antibody to an antigen. Specifically, the rate constants (kₒₙ and k_{off}) and equilibrium dissociation constants are measured using Fab antibody fragments (i.e., univalent) and PCSK9.

The term “k_{off}”, as used herein, refers to the rate constant for dissociation of an antibody from the antibody/antigen complex.

The term “K_{D}”, as used herein, refers to the equilibrium dissociation constant of an antibody-antigen interaction.

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. Exemplary methods and materials are described herein, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. The materials, methods, and examples are illustrative only and not intended to be limiting.

Published information related to anti-PCSK9 antibodies includes the following published applications: PCT/IB2009/053990, published Mar. 18, 2010 as WO 2010/029513, and U.S. patent application No. 12/558312, published Mar. 18, 2010 as US 2010/0068199, each of which is herein incorporated by reference in its entirety.

Treatment with Anti-PCSK9 Antibodies

Provided herein are therapeutic regimens for treatment of disorders characterized by marked elevations of LDL particles in the plasma. The subject therapeutic regimens involve administration of a PCSK9 antagonist antibody. In some embodiments, the subject therapeutic regimens involve administration of a PCSK9 antagonist antibody to a patient who has been receiving stable doses of a statin. The therapeutic regimens disclosed herein provide an effective amount of a PCSK9 antagonist antibody that antagonizes circulating PCSK9 for use in treating or preventing hypercholesterolemia, and/or at least one symptom of dyslipidemia, atherosclerosis, cardiovascular disease, acute coronary syndrome (ACS), or coronary heart disease, in an individual.

Advantageously, the therapeutic regimens disclosed herein result in substantial and durable LDL-C lowering. Preferably, blood cholesterol and/or blood LDL is at least about 10% or 15% lower than before administration. More preferably, blood cholesterol and/or blood LDL is at least about 20, 30, 40, 50, 60, 70 or 80% lower than before administration of the antibody.

### Dosing Regimens

In some embodiments, a dosing regimen comprises administering an initial dose of about 2 mg/kg of the PCSK9 antibody, followed by a maintenance dose of about 2 mg/kg every 4 weeks. In other embodiments, a dosing regimen comprises administering an initial dose of about 4 mg/kg of the PCSK9 antibody, followed by a maintenance dose of about 4 mg/kg every 4 weeks. In other embodiments, a dosing regimen comprises administering an initial dose of about 4 mg/kg of the PCSK9 antibody, followed by a maintenance dose of about 4 mg/kg every 8 weeks. In other embodiments, a dosing regimen comprises administering an initial dose of about 8 mg/kg of the PCSK9 antibody, followed by a maintenance dose of about 8 mg/kg every 8 weeks. In other embodiments, a dosing regimen comprises administering an initial dose of about 12 mg/kg of the PCSK9 antibody, followed by a maintenance dose of about 12 mg/kg every 8 weeks.

In other embodiments, a dosing regimen comprises administering a weekly dose of about 0.25 mg/kg of the PCSK9 antibody. In other embodiments, a dosing regimen comprises administering a weekly dose of about 0.5 mg/kg of the PCSK9 antibody. In other embodiments, a dosing regimen comprises administering a weekly dose of about 1 mg/kg of the PCSK9 antibody. In other embodiments, a dosing regimen comprises administering a weekly dose of about 1.5 mg/kg of the PCSK9 antibody.

However, other dosage regimens may be useful, depending on the pattern of pharmacokinetic decay that the practitioner wishes to achieve. The progress of this therapy is easily monitored by conventional techniques and assays. In preferred embodiments, the initial dose and the first subsequent and additional subsequent doses are separated in time from each other by at least four weeks. The dosing regimen (including the PCSK9 antagonist(s) used) can vary over time.

Generally, for administration of PCSK9 antibodies, an initial candidate dosage can be about 0.3 mg/kg to about 18 mg/kg of the PCSK9 antagonist antibody. For the purpose of the present invention, a typical dosage might range from about any of about 3 μg/kg to 30 μg/kg to 300 μg/kg to 3 mg/kg, to 30 mg/kg, to 100 mg/kg or more, depending on the factors mentioned above. For example, dosage of about 0.3 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 10.5 mg/kg, about 11 mg/kg, about 11.5 mg/kg, about 12 mg/kg, about 12.5 mg/kg, about 13 mg/kg, about 13.5 mg/kg, about 14 mg/kg, about 14.5 mg/kg, about 15 mg/kg, about 15.5 mg/kg, about 16 mg/kg, about 16.5 mg/kg, about 17 mg/kg, about 17.5 mg/kg, about 18 mg/kg, about 18.5 mg/kg, about 19 mg/kg, about 19.5 mg/kg, about 20 mg/kg, about 20.5 mg/kg, about 21 mg/kg, about 21.5 mg/kg, about 22 mg/kg, about 22.5 mg/kg, about 23 mg/kg, about 23.5 mg/kg, about 24 mg/kg, about 24.5 mg/kg, and about 25 mg/kg may be used. For repeated administrations over several days or longer, depending on the condition, the treatment is sustained until a desired suppression of symptoms occurs or until sufficient therapeutic levels are achieved, for example, to reduce blood LDL levels.

An exemplary dosing regimen comprises administering an initial dose of about 0.25 mg/kg, about 0.5 mg/kg, about 1 mg/kg, about 1.5 mg/kg, about 2 mg/kg, about 2.5 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, or about 18 mg/kg, followed by a maintenance dose of about 0.25 mg/kg, about 0.5 mg/kg, about 1 mg/kg, about 1.5 mg/kg, about 2 mg/kg, about 2.5 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, or about 18 mg/kg of the PCSK9 antibody. In some embodiments, the maintenance dose is administered weekly. In some embodiments, the maintenance dose is administered every other week. In some embodiments, the maintenance dose is administered about every three weeks. In some embodiments, the maintenance dose is administered about every four weeks. In some embodiments, the maintenance dose is administered about every five weeks. In some embodiments, the maintenance dose is administered about every six weeks. In some embodiments, the maintenance dose is administered about every seven weeks. In some embodiments, the maintenance dose is administered about every eight weeks. In preferred embodiments, the initial dose and the first subsequent and additional subsequent doses are separated in time from each other by at least about four weeks. In some embodiments, the maintenance dose is administered monthly. In other embodiments, a fixed dose may be used. For example, a PCSK9 antagonist antibody dose of about 0.25 mg, about 0.3 mg, about 0.5 mg, about 1 mg, about 1.5 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 99 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 199 mg, about 198 mg, about 199 mg, about 200 mg, about 250, about 300, about 350, about 400, about 450, or about 500 mg may be used. In some embodiments, the fixed doses is administered subcutaneously or intravenously.

PCSK9 antagonist antibodies can be administered according to one or more dosing regimens disclosed herein to an individual on stable doses of a statin. The stable doses can be, for example without limitation, a daily dose or an every-other-day dose of a statin. A variety of statins known to those of skill in the art, and include, for example without limitation, atorvastatin, simvastatin, lovastatin, pravastatin, rosuvastatin, fluvastatin, cerivastatin, mevastatin, pitavastatin, and statin combination therapies. Non-limiting examples of statin combination therapies include atorvastatin plus amlodipine (CADUET™), simvastatin plus ezetimibe (VYTORIN™), lovastatin plus niacin (ADVICOR™), and simvastatin plus niacin (SIMCOR™).

In some embodiments, an individual has been on stable doses of a statin for at least one, two, three, four, five or six weeks prior to administration of an initial dose of PCSK9 antagonist antibody. Preferably, the individual on stable doses of a statin has a fasting LDL-C greater than or equal to about 70 mg/dL prior to administration of an initial dose of PCSK9 antagonist antibody. In some embodiments, the individual on stable doses of a statin has a fasting LDL-C greater than or equal to about 80, 90, 100, 110, 120, 130, 140, 150, 160, 170, 180, 190 or 200 mg/dL prior to administration of an initial dose of PCSK9 antagonist antibody.

For the purpose of the present invention, a typical statin dose might range from about 1 mg to about 80 mg, depending on the factors mentioned above. For example, a statin dose of about 0.3 mg, about 0.5 mg, about 1 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, or about 80 mg may be used.

In preferred embodiments, a dose of 40 mg or 80 mg atorvastatin is used. In other embodiments, a dose of 20 mg or 40 mg rosuvastatin is used. In other embodiments, a dose of 40 mg or 80 mg simvastatin is used.

In some embodiments, a dosing regimen comprises administering to a subject on stable doses of a statin an initial dose of about 2 mg/kg of the PCSK9 antibody, followed by a maintenance dose of about 2 mg/kg about every 4 weeks. In other embodiments, a dosing regimen comprises administering to a subject on stable doses of a statin an initial dose of about 3 mg/kg of the PCSK9 antibody, followed by a maintenance dose of about 3 mg/kg about every 4 weeks. In other embodiments, a dosing regimen comprises administering to a subject on stable doses of a statin an initial dose of about 4 mg/kg of the PCSK9 antibody, followed by a maintenance dose of about 4 mg/kg about every 4 weeks. In other embodiments, a dosing regimen comprises administering to a subject on stable doses of a statin an initial dose of about 5 mg/kg of the PCSK9 antibody, followed by a maintenance dose of about 5 mg/kg about every 4 weeks. In other embodiments, a dosing regimen comprises administering to a subject on stable doses of a statin an initial dose of about 4 mg/kg of the PCSK9 antibody, followed by a maintenance dose of about 4 mg/kg every 8 weeks. In other embodiments, a dosing regimen comprises administering to a subject on stable doses of a statin an initial dose of about 6 mg/kg of the PCSK9 antibody, followed by a maintenance dose of about 6 mg/kg about every 4 weeks. In other embodiments, a dosing regimen comprises administering to a subject on stable doses of a statin an initial dose of about 8 mg/kg of the PCSK9 antibody, followed by a maintenance dose of about 8 mg/kg every 8 weeks. In other embodiments, a dosing regimen comprises administering to a subject on stable doses of a statin an initial dose of about 12 mg/kg of the PCSK9 antibody, followed by a maintenance dose of about 12 mg/kg every 8 weeks.

In other embodiments, a dosing regimen comprises administering to a subject on stable doses of a statin an initial dose of about 200 mg of the PCSK9 antibody subcutaneously, followed by a maintenance dose of about 200 mg about every 4 weeks. In other embodiments, a dosing regimen comprises administering to a subject on stable doses of a statin an initial dose of about 300 mg of the PCSK9 antibody, followed by a maintenance dose of about 300 mg about every 4 weeks. In other embodiments, a dosing regimen comprises administering to a subject on stable doses of a statin an initial dose of about 50 mg of the PCSK9 antibody, followed by a maintenance dose of about 50 mg about every 2 weeks. In other embodiments, a dosing regimen comprises administering to a subject on stable doses of a statin an initial dose of about 100 mg of the PCSK9 antibody, followed by a maintenance dose of about 100 mg about every 2 weeks. In other embodiments, a dosing regimen comprises administering to a subject on stable doses of a statin an initial dose of about 150 mg of the PCSK9 antibody, followed by a maintenance dose of about 150 mg about every 2 weeks.

Another exemplary dosing regimen comprises administering to a subject on stable doses of a statin an initial dose of about 0.25 mg/kg of the PCSK9 antagonist antibody. In some embodiments, the dosing regimen further comprises administering a monthly maintenance dose of about 0.25 mg/kg of the PCSK9 antagonist antibody. Another exemplary dosing regimen comprises administering to a subject on stable doses of a statin an initial dose of about 0.5 mg/kg of the PCSK9 antagonist antibody. In some embodiments, the dosing regimen further comprises administering a monthly maintenance dose of about 0.5 mg/kg of the PCSK9 antagonist antibody. Another exemplary dosing regimen comprises administering to a subject on stable doses of a statin an initial dose of about 1 mg/kg of the PCSK9 antagonist antibody. In some embodiments, the dosing regimen further comprises administering a monthly maintenance dose of about 1 mg/kg of the PCSK9 antagonist antibody. Another exemplary dosing regimen comprises administering to a subject on stable doses of a statin an initial dose of about 1.5 mg/kg of the PCSK9 antagonist antibody. In some embodiments, the dosing regimen further comprises administering a monthly maintenance dose of about 1.5 mg/kg of the PCSK9 antagonist antibody. Another exemplary dosing regimen comprises administering to a subject on stable doses of a statin an initial dose of about 2 mg/kg of the PCSK9 antagonist antibody. In some embodiments, the dosing regimen further comprises administering a monthly maintenance dose of about 2 mg/kg of the PCSK9 antagonist antibody. Another exemplary dosing regimen comprises administering to a subject on stable doses of a statin an initial dose of about 3 mg/kg of the PCSK9 antagonist antibody. Another exemplary dosing regimen comprises administering to a subject on stable doses of a statin an initial dose of about 4 mg/kg of the PCSK9 antagonist antibody. In some embodiments, the dosing regimen further comprises administering a monthly maintenance dose of about 4 mg/kg of the PCSK9 antagonist antibody. Another exemplary dosing regimen comprises administering to a subject on stable doses of a statin an initial dose of about 5 mg/kg of the PCSK9 antagonist antibody. In some embodiments, the dosing regimen further comprises administering a monthly maintenance dose of about 5 mg/kg of the PCSK9 antagonist antibody. Another exemplary dosing regimen comprises administering to a subject on stable doses of a statin an initial dose of about 6 mg/kg of the PCSK9 antagonist antibody. In some embodiments, the dosing regimen further comprises administering a monthly maintenance dose of about 6 mg/kg of the PCSK9 antagonist antibody.

However, other dosage regimens may be useful, depending on the pattern of pharmacokinetic decay that the practitioner wishes to achieve. The progress of this therapy is easily monitored by conventional techniques and assays. In preferred embodiments, the initial dose and the first subsequent and additional subsequent doses are separated in time from each other by at least four weeks. The dosing regimen (including the PCSK9 antagonist(s) used) can vary over time.

### PCSK9 Antagonist Antibodies

A description follows as to an exemplary technique for the production of the antibodies used in accordance with the present invention. The PCSK9 antigen to be used for production of antibodies may be, e.g. full-length human PCSK9, full length mouse PCSK9, and various peptides fragments of PCSK9. Other forms of PCSK9 useful for generating antibodies will be apparent to those skilled in the art.

Monoclonal antibodies were generated by immunizing PCSK9 null mice with recombinant full-length PCSK9 protein. This manner of antibody preparation yielded antagonist antibodies that show complete blocking of PCSK9 binding to LDLR, complete blocking of PCSK9-mediated lowering of LDLR levels in Huh7 cells, and lowering of LDL cholesterol levels in vivo including in mice to levels comparable to that seen in PCSK9−/−mice, as shown in Example 7 of U.S. patent application Ser. No. 12/558312.

As will be appreciated, antibodies for use in the present invention may be derived from hybridomas but can also be expressed in cell lines other than hybridomas. Sequences encoding the cDNAs or genomic clones for the particular antibodies can be used for transformation of suitable mammalian or nonmammalian host cells. Mammalian cell lines available as hosts for expression are well known in the art and include many immortalized cell lines available from the American Type Culture Collection (ATCC), including but not limited to Chinese hamster ovary (CHO) cells, NSO, HeLa cells, baby hamster kidney (BHK) cells, monkey kidney cells (COS), and human hepatocellular carcinoma cells (e.g., Hep G6). Non-mammalian cells can also be employed, including bacterial, yeast, insect, and plant cells. Site directed mutagenesis of the antibody CH6 domain to eliminate glycosylation may be preferred in order to prevent changes in either the immunogenicity, pharmacokinetic, and/or effector functions resulting from non-human glycosylation. The glutamine synthase system of expression is discussed in whole or part in connection with European Patents 616 846, 656 055, and 363 997 and European Patent Application 89303964.4. Further, a dihydrofolate reductase (DHFR) expression system, including those known in the art, can be used to produce the antibody.

In some embodiments, the invention is practiced using the PCSK9 antagonist antibody L1L3. In some embodiments, the invention is practiced using an antibody that recognizes an epitope of PCSK9 that is the same as the epitope that is recognized by antibody L1L3.

In some embodiments, the invention is practiced using an antibody comprising three CDRS from a heavy chain variable region having the amino acid sequence shown in SEQ ID NO: 11 and three CDRS from a light chain variable region having the amino acid sequence shown in SEQ ID NO: 12.

In some embodiments, the invention is practiced using an antibody that specifically binds PCSK9 comprising a VH complementary determining region one (CDR1) having the amino acid sequence shown in SEQ ID NO: 2 (SYYMH), SEQ ID NO: 13 (GYTFTSY), or SEQ ID NO: 14 (GYTFTSYYMH); a VH CDR2 having the amino acid sequence shown in SEQ ID NO: 3 (EISPFGGRTNYNEKFKS) or SEQ ID NO: 15 (ISPFGGR), and/or VH CDR3 having the amino acid sequence shown in SEQ ID NO: 4 (ERPLYASDL), or a variant thereof having one or more conservative amino acid substitutions in said sequences of CDR1, CDR2, and/or CDR3, wherein the variant retains essentially the same binding specificity as the CDR defined by said sequences. Preferably, the variant comprises up to about ten amino acid substitutions and, more preferably, up to about four amino acid substitutions.

In some embodiments, the invention is practiced using an antibody comprising a VL CDR1 having the amino acid sequence shown in SEQ ID NO: 5 (RASQGISSALA), a CDR2 having the amino acid sequence shown in SEQ ID NO: 6 (SASYRYT), and/or CDR3 having the amino acid sequence shown in SEQ ID NO: 7 (QQRYSLWRT), or a variant thereof having one or more conservative amino acid substitutions in said sequences of CDR1, CDR2, and/or CDR3, wherein the variant retains essentially the same binding specificity as the CDR1 defined by said sequences. Preferably, the variant comprises up to about ten amino acid substitutions and, more preferably, up to about four amino acid substitutions.

In some embodiments, the invention is practiced using an antibody having a heavy chain sequence comprising or consisting of SEQ ID NO: 8 or 10 and a light chain sequence comprising or consisting of SEQ ID NO: 9.

In some embodiments, the invention is practiced using an antibody having a heavy chain variable region comprising or consisting of the amino acid sequence shown in SEQ ID NO: 11 and a light chain variable region comprising or consisting of the amino acid sequence shown in SEQ ID NO: 12.

In some embodiments, the invention is practiced using an antibody that recognizes an epitope on human PCSK9 comprising amino acid residues 153-155, 194, 195, 197, 237-239, 367, 369, 374-379 and 381 of the PCSK9 amino acid sequence of SEQ ID NO: 1. Preferably, the antibody epitope on human PCSK9 does not comprise one or more of amino acid residues 71, 72, 150-152, 187-192, 198-202, 212, 214-217, 220-226, 243, 255-258, 317, 318, 347-351, 372, 373, 380, 382, and 383 of the PCSK9 amino acid sequence of SEQ ID NO: 1.

In some embodiments, the invention is practiced using an antibody that recognizes a first epitope of PCSK9 that is the same as or overlaps with a second epitope that is recognized by a monoclonal antibody selected from the group consisting of 5A10, which is produced by a hybridoma cell line deposited with the American Type Culture Collection and assigned accession number PTA-8986; 4A5, which is produced by a hybridoma cell line deposited with the American Type Culture Collection and assigned accession number PTA-8985; 6F6, which is produced by a hybridoma cell line deposited with the American Type Culture Collection and assigned accession number PTA-8984, and 7D4, which is produced by a hybridoma cell line deposited with the American Type Culture Collection and assigned accession number PTA-8983. In preferred embodiments, the invention is practiced using the PCSK9 antagonist antibody L1L3 (see, PCT/IB2009/053990, published March 18, 2010 as WO 2010/029513, and U.S. patent application Ser. No. 12/558312, published Mar.18, 2010 as US 2010/0068199).

Preferably, the variant comprises up to about twenty amino acid substitutions and more preferably, up to about eight amino acid substitutions. Preferably, the antibody rther comprises an immunologically inert constant region, and/or the antibody has an isotype that is selected from the group consisting of IgG₂, IgG₄, IgG_{2Δa}, IgG_{4Δb}, IgG_{4Δc}, IgG₄ S228P, IgG_{4Δb} S228P and IgG_{4Δc} S228P. In another preferred embodiment, the constant region is aglycosylated Fc.

The antibodies useful in the present invention can encompass monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion (e.g., a domain antibody), human antibodies, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies).

In some embodiments, the PCSK9 antagonist antibody is a monoclonal antibody. The PCSK9 antagonist antibody can also be humanized. In other embodiments, the antibody is human.

In some embodiments, the antibody comprises a modified constant region, such as a constant region that is immunologically inert, that is, having a reduced potential for provoking an immune response. In some embodiments, the constant region is modified as described in Eur. J. Immunol., 1999, 29:2613-2624; PCT Publ. No. W099/58572; and/or UK Patent Application No. 9809951.8. The Fc can be human IgG₂ or human IgG₄. The Fc can be human IgG₂ containing the mutation A330P331 to S330S331 (IgG_{2Δa}), in which the amino acid residues are numbered with reference to the wild type IgG2 sequence. Eur. J. Immunol., 1999, 29:2613-2624. In some embodiments, the antibody comprises a constant region of IgG₄ comprising the following mutations (Armour et al., 2003, Molecular Immunology 40 585-593): E233F234L235 to P233V234A235 (IgG_{4Δc}, in which the numbering is with reference to wild type IgG4. In yet another embodiment, the Fc is human IgG₄ E233F234L235 to P233V234A235 with deletion G236 (IgG_{4Δb}). In another embodiment the Fc is any human IgG₄ Fc (IgG₄, IgG_{4Δb} or IgG_{4Δc}) containing hinge stabilizing mutation S228 to P228 (Aalberse et al., 2002, Immunology 105, 9-19). In another embodiment, the Fc can be aglycosylated Fc.

In some embodiments, the constant region is aglycosylated by mutating the oligosaccharide attachment residue (such as Asn297) and/or flanking residues that are part of the glycosylation recognition sequence in the constant region. In some embodiments, the constant region is aglycosylated for N-linked glycosylation enzymatically. The constant region may be aglycosylated for N-linked glycosylation enzymatically or by expression in a glycosylation deficient host cell.

In some embodiments, more than one antagonist antibody may be present. At least one, at least two, at least three, at least four, at least five different, or more antagonist antibodies and/or peptides can be present. Generally, those PCSK9 antagonist antibodies or peptides may have complementary activities that do not adversely affect each other. A PCSK9 antagonist antibody can also be used in conjunction with other PCSK9 antagonists or PCSK9 receptor antagonists. For example, one or more of the following PCSK9 antagonists may be used: an antisense molecule directed to a PCSK9 (including an anti-sense molecule directed to a nucleic acid encoding PCSK9), a PCSK9 inhibitory compound, and a PCSK9 structural analog. A PCSK9 antagonist antibody can also be used in conjunction with other agents that serve to enhance and/or complement the effectiveness of the agents.

With respect to all methods described herein, reference to PCSK9 antagonist antibodies also include compositions comprising one or more additional agents. These compositions may further comprise suitable excipients, such as pharmaceutically acceptable excipients including buffers, which are well known in the art. The present invention can be used alone or in combination with other conventional methods of treatment.

The PCSK9 antagonist antibody can be administered to an individual via any suitable route. It should be apparent to a person skilled in the art that the examples described herein are not intended to be limiting but to be illustrative of the techniques available. Accordingly, in some embodiments, the PCSK9 antagonist antibody is administered to an individual in accord with known methods, such as intravenous administration, e.g., as a bolus or by continuous infusion over a period of time, by intramuscular, intraperitoneal, intracerebrospinal, transdermal, subcutaneous, intra-articular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively,

PCSK9 antagonist antibody can be aerosolized using a fluorocarbon formulation and a metered dose inhaler, or inhaled as a lyophilized and milled powder.

In one embodiment, a PCSK9 antagonist antibody is administered via site-specific or targeted local delivery techniques. Examples of site-specific or targeted local delivery techniques include various implantable depot sources of the PCSK9 antagonist antibody or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publ. No. WO 00/53211 and U.S. Pat. No. 5,981,568.

Various formulations of a PCSK9 antagonist antibody may be used for administration. In some embodiments, the PCSK9 antagonist antibody may be administered neat. In some embodiments, PCSK9 antagonist antibody and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy, 20th Ed., Mack Publishing (2000).

These agents can be combined with pharmaceutically acceptable vehicles such as saline, Ringer's solution, dextrose solution, and the like. The particular dosage regimen, i.e., dose, timing and repetition, will depend on the particular individual and that individual's medical history.

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Liposomes containing the PCSK9 antagonist antibody are prepared by methods known in the art, such as described in Epstein, et al., 1985, Proc. Natl. Acad. Sci. USA 82:3688; Hwang, et al., 1980, Proc. Natl Acad. Sci. USA 77:4030; and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

The formulations to be used for in vivo administration must be sterile. This is readily accomplished by, for example, filtration through sterile filtration membranes. Therapeutic PCSK9 antagonist antibody compositions are generally placed into a container having a sterile access port, for example, an intravenous solution bag or vial having a stopper pierceable by a hypodermic injection needle.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 pm, particularly 0.1 and 0.5 pm, and have a pH in the range of 5.5 to 8.0.

The emulsion compositions can be those prepared by mixing a PCSK9 antagonist antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

Polynucleotides encoding the heavy and light chain variable regions of antibody L1L3 were deposited in the American Type Culture Collection (ATCC), 10801 University Boulevard, Manassas, Va. 90110, U.S.A., on Aug. 25, 2009. The L1L3 heavy chain variable region polynucleotide deposit was assigned ATCC Accession No. PTA-10302, and the L1L3 light chain variable region polynucleotide deposit was assigned ATCC Accession No. PTA-10303. The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

### EXAMPLES

The following examples are meant to illustrate the methods and materials of the present invention. Suitable modifications and adaptations of the described conditions and parameters normally encountered in the art that are obvious to those skilled in the art are within the spirit and scope of the present invention.

### Example 1

### Treatment with a Humanized PCSK9 Antagonist Antibody L1L3 is Effective for Reducing in Serum Cholesterol and LDL Cholesterol Levels

This example illustrates efficacy of a humanized PCSK9 antagonist antibody, L1L3, in reducing serum cholesterol and LDL cholesterol levels in animal models.

L1L3 is a humanized (<5% murine residues) monoclonal antibody that binds to secreted PCSK9, effectively prevents its down-regulation of LDLR, leading to improved LDL clearance in serum and reduction of LDL-C.

When 10 mg/kg of L1L3 was administered as a single intraperitoneal (IP) dose to C57BU6 mice fed a normal diet (n=10), serum cholesterol levels were reduced to 47 mg/dL (37% reduction) compared to 75 mg/dL in saline treated controls 48 hours post treatment and 44 mg/dL (47% reduction) compared to 83 mg/dL in control animals 4 days post-treatment. Serum cholesterol levels recovered to 69 mg/dL by day 7 post-treatment.

L1L3 was administered as a single IP dose at 0, 0.1, 1, 10 and 80 mg/kg (n=6/group) in a dose-response experiment in Sprague-Dawley rats fed a normal diet. Serum cholesterol levels were dose-dependently reduced, with maximum effect of 50% seen at 10 and 80 mg/kg 48 hours post dosing. The duration of the cholesterol repression was also dose dependent, ranging from 1 to 21 days. Both the magnitude and duration of the cholesterol-lowering effect of L1L3 correlated with drug exposure. Non-fasting serum triglyceride levels also dose-dependently increased, with a maximum increase of approximately three fold at 80 mg/kg, and a time course correlated with drug exposure. Since similar effects of L1L3 on serum triglyceride levels were not observed in other species such as mice and non-human primate (see below), and changes in blood triglyceride levels were not reported in humans harboring PCSK9 mutations (Abifadel et al., 2003, Nat. Genet., 34:154-156; Cohen et al., 2005, Nat. Genet. 37:161-165; Zhao et al., 2006, Am. J. Hum. Genet. 79:514-523), the increase in serum triglyceride levels caused by L1L3 treatment appears to be a species-specific phenomenon in rat.

In cynomolgus monkeys, fed a normal diet, L1L3 was administered as a single IV dose at 0.1, 1, 3 and 10 mg/kg (n=4/group). Administration of 0.1 mg/kg L1L3 caused a transient 50% drop in LDL-C levels at day 2 and quickly recovered by day 5. One (1) mg/kg dosing reached a maximum effect of 71% reduction in LDL-C on day 5, and began to recover immediately thereafter, reaching pre-dose levels by day 14. Three (3) mg/kg dosing reached a maximum effect of 72% reduction in LDL-C by day 7, levels began to recover by day 13, and returned to baseline by day 22. Ten (10) mg/kg dosing maintained the 70% reduction in LDL-C levels until day 21 post-dosing, and animals fully recovered by day 31. Both the magnitude and duration of the LDL -C lowering effect of L1L3 correlated with drug exposure. HDL-C levels were not affected by L1L3 treatment in all dose groups.

The monkeys in the 3 mg/kg dose group (n=4) were also given two additional IV doses of 3 mg/kg L1L3 on study days 42 and 56 (2-weeks apart). These two additional doses again lowered LDL-C and kept LDL-C levels below 50% for 4 weeks. LDL-C levels returned to normal two weeks later. Serum HDL-C levels remained unchanged.

PK studies were conducted by a single bolus i.v. injection of 0.1, 1.0, 3.0, 10.0 and 100.0 mg/kg of L1L3 in cynomolgus monkeys and total antibody concentration was measured. The estimated β-phase half-life for L1L3 was 0.67 days at a single dose of 0.1 mg/kg, and increased to 1.91, 2.33, 3.49 and 5.25 days at 1.0, 3.0, 10.0 and 100.0 mg/kg, respectively. Thus, in cynomolgus monkeys, L1L3 demonstrated a dose-dependent and non-linear shortening of half-life consistent with antigen mediated degradation and seen with antibody therapeutics having membrane-associated antigens.

In summary, L1L3 binds to and antagonizes serum PCSK9 function, resulting in rapid and significant reduction in serum cholesterol and LDL cholesterol levels in animal models.

### Example 2

### Pharmacokinetics and Pharmacodynamics Following Single, Escalating, Intravenous Doses of PCSK9 Antagonist Antibody L1L3

This example illustrates a clinical trial study to evaluate pharmacokinetics and pharmacodynamics following single, escalating, intravenous doses of a humanized PCSK9 antagonist antibody, L1L3, in otherwise healthy human subjects who were candidates for cholesterol lowering therapy. Administration of L1L3 resulted in a lowering of LDL-C in all dosage groups evaluated.

The study entailed a randomized, placebo-controlled, ascending, single dose study of L1L3. The subjects, investigator, and site personnel (except site personnel responsible for drug preparation) were blinded to treatment assignments, as was the CRO designee; while the Sponsor clinical research team was unblinded. The study was conducted in 6 planned cohorts of 8 subjects per cohort in an effort to seek a maximum tolerated dose or MTD (total of approximately 48 subjects). Within each cohort subjects were randomized to either L1L3 or placebo (3:1 allocation ratio). Doses were administered following an overnight fast as an intravenous infusion over 60 minutes. Infusion rates were carefully controlled by an infusion device per protocol. Infusions will be administered as a single infusion over 60 minutes.

Dosing was as illustrated below in Table 1:

TABLE 1

| | | | Number of |
|---|---|---|---|
| | | | Subjects |
| Cohort | Dose L1L3 | | Dosed |
| 1 | 0.3 | mg/kg | 6 |
| | Placebo | | 2 |
| 2 | 1.0 | mg/kg | 6 |
| | Placebo | | 2 |
| 3 | 3.0 | mg/kg | 6 |
| | Placebo | | 2 |
| 4 | 6.0 | mg/kg | 6 |
| | Placebo | | 2 |
| 5 | 12 | mg/kg | 6 |
| | Placebo | | 2 |
| 6 | 18 | mg/kg | 6 |

| | |
|---|---|
| Placebo | 2 |

The dosing schedule was adjusted to allow administration of lower, intermediate, or higher doses to obtain a maximum tolerated dose and no effect dose. Each subject enrolled into the study, regardless of cohort assignment, received only one dose of study drug during their study participation. All patients were observed for safety for an additional 21 days (total 28 days) prior to their study completion.

The primary PK endpoints of the study were AUC_{(0-t[last])}, Tₘₐₓ, and Cₘₐₓ of L1L3. Secondary PK endpoints included terminal elimination half-life (T_{1/2}), Clearance (CL), Volume in steady state (Vss), and AUC_{(0-∞)} of L1L3. Change in serum lipids (total cholesterol, LDL, HDL, Triglycerides, Non-HDL-C and Apoprotein B) were assessed. Screening occurred within 28 days of the dose for each subject. Subjects received a single dose of L1L3 on Day 0, with daily PK and safety assessments through confinement period (study Days-1, 0, and 1) as well as on days 4, 7, 14, 21, 28 and, depending upon initial PK findings, after day 28.

Inclusion criteria for the study were as follows: healthy, ambulatory, males and/or females (females will be women of non-childbearing potential) between the ages of 18 and 70 years, inclusive; baseline total cholesterol 200 mg/dl, baseline LDL 130 mg/dl; body mass index (BMI) of 18.5 to 35 kg/m² BMI 18.5 to 35, and body weight 150 kg, inclusive; evidence of a personally signed and dated informed consent document indicating that the subject (or a legally acceptable representative) has been informed of all pertinent aspects of the trial; and willing and able to comply with scheduled visits, treatment plan, laboratory tests, and other trial procedures.

Exclusion criteria for the study were as follows: evidence or history of clinically significant hematological, renal, endocrine, pulmonary, gastrointestinal, cardiovascular, hepatic, psychiatric, neurologic, or allergic disease (including drug allergies, but excluding untreated, asymptomatic, seasonal allergies at time of dosing); secondary hyperlipidemia; subjects should not have taken other prescription medications for at least 1 week prior to dosing. If patients have received lipid lowering medications these drugs should have been discontinued for an adequate period of time to allow return of serum lipids to pretreatment levels; history of febrile illness within 5 days prior to dosing; history of stroke or transient ischemic attack; history of myocardial infarction within the past year; a positive urine drug screen; history of regular alcohol consumption exceeding 7 drinks/week for females or 14 drinks/week for men (1 drink=5 ounces (150 mL) of wine or 12 ounces (360 mL) of beer or 1.5 ounces (45 mL) of hard liquor) within 6 months of screening; treatment with an investigational drug within 30 days or 5 half-lives (whichever is longer) preceding the first dose of trial medication; 12-lead ECG demonstrating QTc >450 msec at screening; pregnant or nursing females; women of childbearing potential; blood donation of approximately 1 pint (500 mL) within 56 days prior to dosing; history of sensitivity to heparin or heparin-induced thrombocytopenia (if heparin is used to flush intravenous catheters; other severe acute or chronic medical or psychiatric condition or laboratory abnormality that may increase the risk associated with study participation or investigational product administration or may interfere with the interpretation of study results and, in the judgment of the Investigator, would make the subject inappropriate for entry into this study.

Subjects were randomized into the study provided they have satisfied all subject selection criteria. A computer-generated randomization schedule was used to assign subjects to the treatment sequences.

For dose escalation, the decision to proceed to a higher dose of L1L3 was made by the Sponsor and the Investigator after review of the available safety and tolerability data from all cohort subjects followed for at least 7 days following administration of the previous dose level.

L1L3 drug product (100 mg) was provided in sterile, liquid form at a concentration of 10 mg/mL in a glass vial for intravenous (IV) administration, with a rubber stopper and aluminum seal. Each vial contained 10 mL (extractable volume) of L1L3 at a concentration of 10 mg/mL and a pH of 5.5. L1L3 and placebo were prepared according to the Dosage and Administration Instructions in the Pharmacy Manual that will be provided to the site. Drug was prepared by qualified unblinded site personnel and dispensed in a blinded fashion to the patient and immediate study staff. L1L3 was administered by rate controlled intravenous infusion over approximately 60 minutes in accordance with the Dosage Administration Instructions (DAI) located in the Pharmacy Manual and Study Reference Guide.

Study Protocol

Day-1: Subjects were assigned a randomization number and admitted to the Clinical Research Unit at least 12 hours prior to the start of Day 0 activities and were required to remain in the Clinical Research Unit (CRU) until completion of procedures on Day 1. Subject began fasting in the evening at least 10 hours prior to scheduled Lipid Panel for Day 0. The following procedures were completed: reviewed changes in medical history since screening; reviewed changes in concomitant medications since screening; reviewed history of drug, alcohol, and tobacco use since screening; assessed symptoms by spontaneous reporting of adverse events and by asking the subjects to respond to a non-leading question such as “How do you feel?”; physical examination, including weight; urine drug screen; obtained supine vital signs; obtained triplicate 12-lead ECGs approximately 2-4 minutes apart

Day 0: Prior to dosing, the following procedures were completed: collected fasting lipid profile after at least a 10-hour fast (total cholesterol, LDL, HDL, Non-HDL Cholesterol, Apo B and triglycerides); collected samples for routine and additional laboratory tests: hematology; chemistry; coagulation, amylase; urinalysis; collected sample for pre-dose PK; collected sample for PCSK9 levels/PD markers of interest; collected sample for Anti-L1L3 antibodies; reviewed changes in concomitant medications since screening; assessed symptoms by spontaneous reporting of adverse events and by asking the subjects to respond to a non-leading question such as “How do you feel?”; obtained supine vital signs; administered Study Drug Infusion according to Pharmacy Manual Instructions.

After dosing, the following procedures were completed: obtained triplicate 12-lead ECGs approximately 2-4 minutes apart beginning within 10 minutes of end of infusion (EOI); obtained supine vital signs at EOI; collected blooded sample for PK analysis at EOI, and the following timepoints post infusion (i.e. EOI+ the following timepoints): 60 minutes, 120 min., and 360 min.

Day 1: The following procedures were completed: collected blood sample for PK analysis at 1440 min (24 hours) +/−30 min post dose; performed abbreviated physical exam; collected fasting lipid profile after at least a 10-hour fast (total cholesterol, LDL, HDL, Non-HDL Cholesterol, Apo B and triglycerides); collected sample for PCSK9 levels/PD markers of interest; assessed symptoms by spontaneous reporting of adverse events and by asking the subjects to respond to a non-leading question such as “How do you feel?”; reviewed changes in concomitant medications since screening; obtained supine vital signs; discharged from CRU.

Day 4: The following procedures were completed: collected samples for routine laboratory tests: hematology; chemistry; and urinalysis; collected fasting lipid profile after at least a 10-hour fast (total cholesterol, LDL, HDL, Non-HDL Cholesterol, Apo B and triglycerides); collected single blood sample for PK analysis; collected sample for PCSK9 levels/PD markers of interest; assessed symptoms by spontaneous reporting of adverse events and by asking the subjects to respond to a non-leading question such as “How do you feel?”; reviewed changes in concomitant medications since screening; obtained supine vital signs

Day 7: The following procedures were completed: performed abbreviated physical exam; collected samples for routine and additional laboratory tests: hematology; chemistry; coagulation, amylase; urinalysis; collected fasting lipid profile after at least a 10-hour fast (total cholesterol, LDL, HDL, Non-HDL Cholesterol, Apo B and triglycerides); collected single blood sample for PK analysis; collected sample for PCSK9 levels/PD markers of interest; collected sample for Anti-L1L3 antibodies; assessed symptoms by spontaneous reporting of adverse events and by asking the subjects to respond to a non-leading question such as “How do you feel?”; reviewed changes in concomitant medications since screening; reviewed history of drug, alcohol, and tobacco use since screening; obtained supine vital signs; obtained triplicate 12-lead ECGs approximately 2-4 minutes apart.

Day 14: The following procedures were completed: performed abbreviated physical exam; collected samples for routine and additional laboratory tests: hematology; chemistry; coagulation, amylase; urinalysis; collected fasting lipid profile after at least a 10-hour fast (total cholesterol, LDL, HDL, Non-HDL Cholesterol, Apo B and triglycerides); collected single blood sample for PK analysis; collected sample for PCSK9 levels/PD markers of interest; collected sample for Anti-L1L3 antibodies; assessed symptoms by spontaneous reporting of adverse events and by asking the subjects to respond to a non-leading question such as “How do you feel?”; reviewed changes in concomitant medications since screening; reviewed history of drug, alcohol, and tobacco use since screening; obtained supine vital signs.

Day 21: The following procedures were completed: performed abbreviated physical exam; collected samples for routine and additional laboratory tests: hematology; chemistry; coagulation, amylase; urinalysis; collected fasting lipid profile after at least a 10-hour fast (total cholesterol, LDL, HDL, Non-HDL Cholesterol, Apo B and triglycerides); collected single blood sample for PK analysis; collected sample for PCSK9 levels/PD markers of interest; collected sample for Anti-L1L3 antibodies; assessed symptoms by spontaneous reporting of adverse events and by asking the subjects to respond to a non-leading question such as “How do you feel?”; reviewed changes in concomitant medications since screening; reviewed history of drug, alcohol, and tobacco use since screening; obtained supine vital signs.

Day 28: The following procedures were completed: performed full physical exam; obtained subject's weight; collected samples for routine and additional laboratory tests: hematology; chemistry; coagulation, amylase; urinalysis; collected fasting lipid profile after at least a 10-hour fast (total cholesterol, LDL, HDL, Non-HDL Cholesterol, Apo B and triglycerides); collected single blood sample for PK analysis; collected sample for PCSK9 levels/PD markers of interest; collected sample for Anti-L1L3 antibodies; assessed symptoms by spontaneous reporting of adverse events and by asking the subjects to respond to a non-leading question such as “How do you feel?”; reviewed changes in concomitant medications since screening; reviewed history of drug, alcohol, and tobacco use since screening; obtained supine vital signs; obtained triplicate 12-lead ECGs approximately 2-4 minutes apart.

Additional Follow-up for Prolonged PK: The following procedures were completed when applicable: performed abbreviated physical ; collected samples for routine and additional laboratory tests: hematology; chemistry; coagulation, amylase; urinalysis; collected fasting lipid profile after at least a 10-hour fast (total cholesterol, LDL, HDL, Non-HDL Cholesterol, Apo B and triglycerides); collected single blood sample for PK analysis; collected sample for PCSK9 levels/PD markers of interest; collected sample for Anti-L1L3 antibodies; assessed symptoms by spontaneous reporting of adverse events and by asking the subjects to respond to a non-leading question such as “How do you feel?”; reviewed changes in concomitant medications since screening; reviewed history of drug, alcohol, and tobacco use since screening; obtained supine vital signs; obtained triplicate 12-lead ECGs approximately 2-4 minutes apart.

Total blood sampling volume for individual patients was approximately 183-210 mL. Plasma samples for analysis of L1L3 levels were collected before dosing on Day 0, at termination of infusion, and at 60, 120, 360 and 1440 minutes (24-hours) after infusion ends. In addition, single PK samples were obtained on Days 4, 7, 14, 21, 28 and additional PK follow-up visit (if applicable). One sample was drawn at each time point.

Blood samples for assessment of PCSK9 levels and other experimental pharmcaodynamic markers of interest were obtained pre-dose on Day 0 and Days 1, 4, 7, 14, 21, 28 and additional follow-up visit if applicable.

Collection of fasting lipid profile was performed after at least a 10-hour fast (total cholesterol, LDL, HDL, Non-HDL Cholesterol, Apo B and triglycerides).

Study Results

L1L3 PK NCA Results: The median half-life of L1L3 administered at 0.3 mg/kg was 2.71 days. The median half-life of L1L3 administered at 1 mg/kg was 4.77 days. The median half-life of L1L3 administered at 3 mg/kg was 8.1 days. The median half-life of L1L3 administered at 6 mg/kg was 7.75 days. The median half-life of L1L3 administered at 12 mg/kg was 12.24 days. The median half-life of L1L3 administered at 18 mg/kg was 11.76 days. The L1L3 PK concentration-time profiles were multi-phasic and consistent with target-mediated drug disposition. However, the half-life of L1L3 in human subjects is unexpectedly and significantly longer than the half-life of L1L3 in cynomologus monkeys (i.e., 1.91, 2.33, 3.49 and 5.25 days at 1.0, 3.0, 10.0 and 100.0 mg/kg, respectively, in cynomologus monkeys (see, Example 1)). The mean rate of drug clearance (CI) for L1L3 administered at 0.3, 1, 3, 6, 12 and 18 mg/kg was 8.70, 6.58, 4.54, 4.33, 3.28 and 3.85 mL/Day/kg, respectively. The PK NCA results from this study are summarized in Table 2 below. In columns 2-7 of the table, the top value indicates the mean, and the bottom value is the median.

TABLE 2
PK NCA Results

| | | | Half- | Cl | | |
|---|---|---|---|---|---|---|
| DOSE | Cmax | Tmax | life | (mL/Day/ | Vss | AUC_{(0-∞)} |
| (mg/kg) | (ng/mL) | (Day) | (Day) | kg) | (mL/kg) | (Day · ng/mL) |
| 0.3 | 10319.67 | 0.083 | 2.74 | 8.70 | 31.77 | 34997.88 |
| | 10537.50 | 0.06 | 2.71 | 8.92 | 30.74 | 33748.15 |
| 1 | 29251.83 | 0.063 | 4.80 | 6.58 | 41.59 | 156399.94 |
| | 28231.50 | 0.06 | 4.77 | 6.00 | 42.34 | 166736.58 |
| 3 | 96711.50 | 0.049 | 8.74 | 4.54 | 49.06 | 709485.10 |
| | 100620.5 | 0.04 | 8.1 | 4.12 | 48.94 | 728278.54 |
| 6 | 175854.33 | 0.056 | 8.36 | 4.33 | 60.45 | 1446945.71 |
| | 177485 | 0.04 | 7.75 | 4.65 | 61.33 | 1289916.44 |
| 12 | 353960.17 | 0.090 | 20.53 | 3.28 | 72.25 | 3768691.17 |
| | 357671.00 | 0.08 | 12.24 | 3.36 | 57.52 | 3599992.39 |
| 18 | 532449.17 | 0.090 | 12.97 | 3.85 | 65.46 | 4812012.99 |
| | 560463.50 | 0.08 | 11.76 | 3.71 | 60.83 | 4857618.28 |

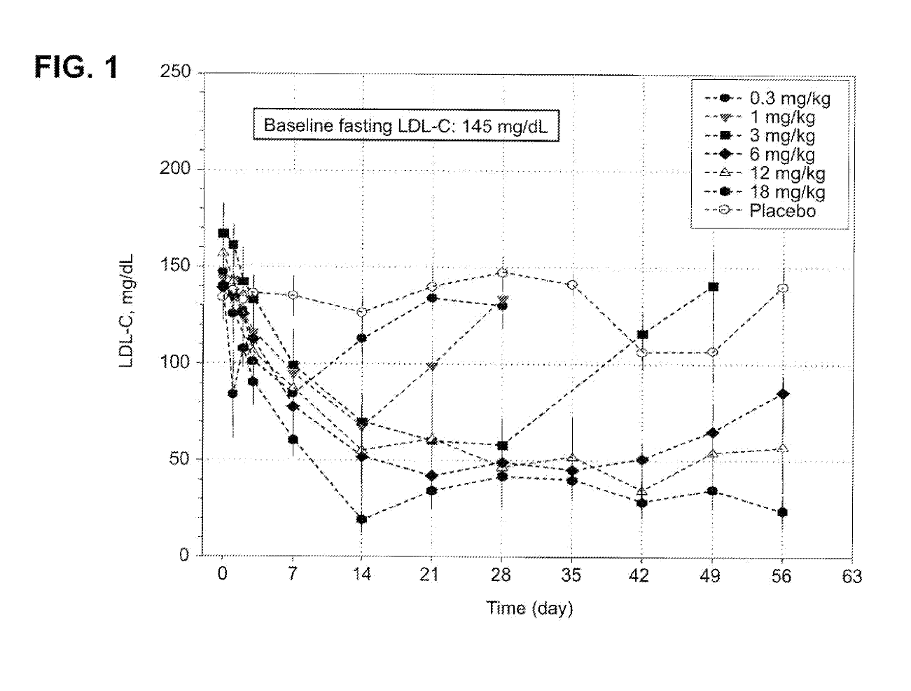

Treatment with L1L3 resulted in substantial and durable dose-dependent fasting LDL-cholesterol (LDL-C) lowering. The LDL-C vs. time profiles are shown in FIG. 1. The baseline fasting LDL-C was about 145 mg/dL. At day 7 post-dosing, LDL-C levels in subjects treated with a single 0.3, 1, 3, 6, 12, or 18 mg/kg dose of L1L3 were between 50 and 100 mg/dL. In contrast, LDL-C levels in subjects administered placebo remained generally about baseline. By day 14 post-dosing, LDL-C levels in subjects treated with 1, 3, 6, 12, or 18 mg/kg L1L3 were about 70 mg/dL or lower. By day 14 post-dosing, subjects treated with 6 mg/kg or 12 mg/kg L1L3 had LDL-C levels of about 55 mg/dL, and subjects treated with 18 mg/kg L1L3 had LDL-C levels of about 20 mg/dL. LDL-C levels in subjects treated with a single 12 mg/kg dose of L1L3 remained at or below about 60 mg/dL until at least about 57 days post-dosing (end of study). LDL-C levels in subjects treated with a single 18 mg/kg dose of L1L3 remained below 50 mg/dL until at least about 57 days post-dosing. LDL-C levels in subjects treated with a single 6 mg/kg dose of L1L3 remained below 50 mg/dL for about 42 days post-dosing and below 100 mg/dL until at least about 57 days post-dosing. LDL-C levels in subjects treated with a single 3 mg/kg dose of L1L3 were about 70 mg/dL at day 14 post-dosing, about 60 mg/dL at day 21 post-dosing, and remained below 100 mg/dL until about 36 days post-dosing. LDL-C levels in subjects treated with a single 1 mg/kg dose of L1L3 were about 65 mg/dL at day 14 post-dosing, and remained below 100 mg/dL until about 21 days post-dosing. LDL-C levels in subjects treated with a single 0.3 mg/kg dose of L1L3 were about 85 mg/dL at day 7 post-dosing, and remained below 100 mg/dL until about 10 days post-dosing.

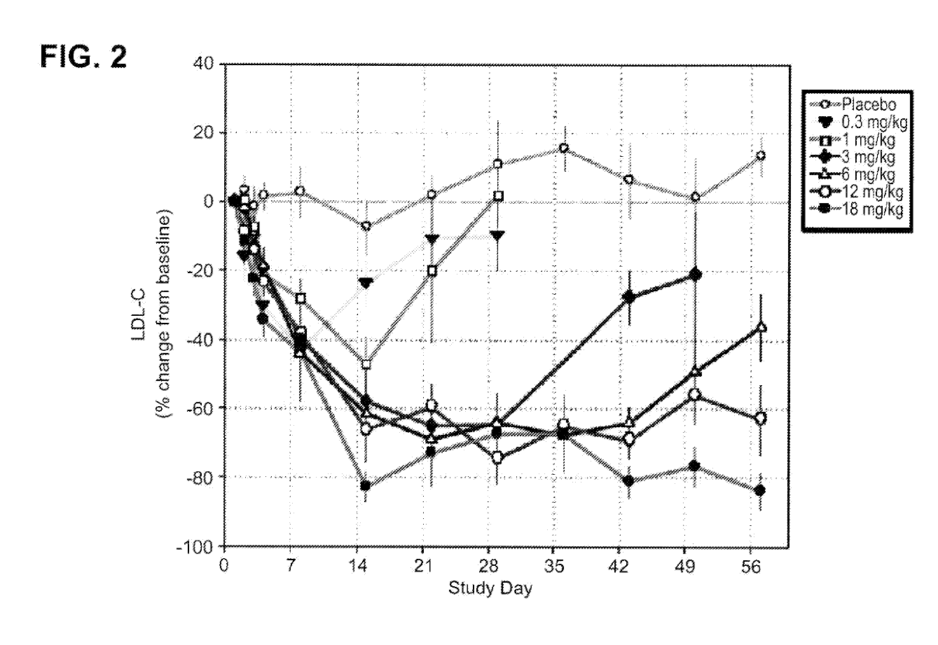

The percentage change from baseline of fasting LDL-C levels in blood is shown in FIG. 2 (data shown are mean +/−SE) and summarized in Table 3 below. In the table, “N” indicates the number of subjects, “mean” indicates the mean percentage change from baseline of fasting LDL-C levels, and “PBO” is placebo.

TABLE 3

| | | | L1L3 | | | | | | | | | | | |
|---|---|---|---|---|---|---|---|---|---|---|---|---|---|---|
| Visit | PBO | | 0.3 mg/kg | | 1 mg/kg | | 3 mg/kg | | 6 mg/kg | | 12 mg/kg | | 18 mg/kg | |
| day | N | mean | N | mean | N | mean | N | mean | N | mean | N | mean | N | mean |
| 1 | 12 | 0.000 | 6 | 0.000 | 6 | 0.000 | 6 | 0.000 | 6 | 0.000 | 6 | 0.000 | 6 | 0.000 |
| 2 | 12 | 3.26 | 6 | −15.8 | 6 | −0.33 | 6 | −1.97 | 6 | −1.44 | 6 | −8.26 | 4 | −11.52 |
| 3 | 12 | −0.5 | 6 | −14.13 | 6 | −9.79 | 6 | −13.20 | 6 | −9.23 | 6 | −14.05 | 6 | −22.43 |
| 4 | 12 | 2.25 | 6 | −30.14 | 6 | −19.14 | 6 | −19.10 | 6 | −18.80 | 5 | −23.23 | 6 | −34.36 |
| 8 | 11 | 8.32 | 6 | −42.86 | 6 | −33.33 | 6 | −39.78 | 6 | −43.77 | 5 | −37.96 | 5 | −43.70 |
| 15 | 11 | −3.24 | 6 | −23.68 | 6 | −50.50 | 6 | −57.93 | 6 | −61.52 | 5 | −66.25 | 5 | −82.89 |
| 22 | 11 | 6.26 | 6 | −11.36 | 6 | −22.40 | 6 | −65.09 | 6 | −68.92 | 5 | −59.79 | 6 | −72.97 |
| 29 | 11 | 11.87 | 6 | −9.12 | 6 | −3.36 | 6 | −64.77 | 6 | −64.19 | 6 | −74.67 | 6 | −67.40 |
| 36 | 5 | 17.38 | | | | | | | 6 | −67.70 | 5 | −65.23 | 4 | −61.47 |
| 43 | 5 | 12.14 | | | | | 3 | −27.56 | 6 | −64.18 | 4 | −69.31 | 3 | −80.21 |
| 50 | 4 | 3.67 | | | | | | | 6 | −49.17 | 4 | −56.08 | | |
| 57 | 4 | 12.08 | | | | | | | 6 | −36.12 | 3 | −63.10 | | |

LDL-C levels in subjects dosed with placebo remained generally at or above baseline, indicated as “0” in FIG. 2. As noted above, the baseline fasting LDL-C was about 145 mg/dL. Administration of 18 mg/kg L1L3 resulted in a percentage change from baseline of up to about 83% (FIG. 2). A single 18 mg/kg L1L3 dose maintained LDL-C levels lower than about 65% below baseline for at least up to 57 days post administration. A single 6 mg/kg or 12 mg/kg L1L3 dose maintained LDL-C levels lower than about 60% below baseline up to 43 days post administration. A single 3 mg/kg L1L3 dose maintained LDL-C levels lower than about 60% below baseline up to 29 days post administration, and lower than 20% below baseline up to 50 days post administration.

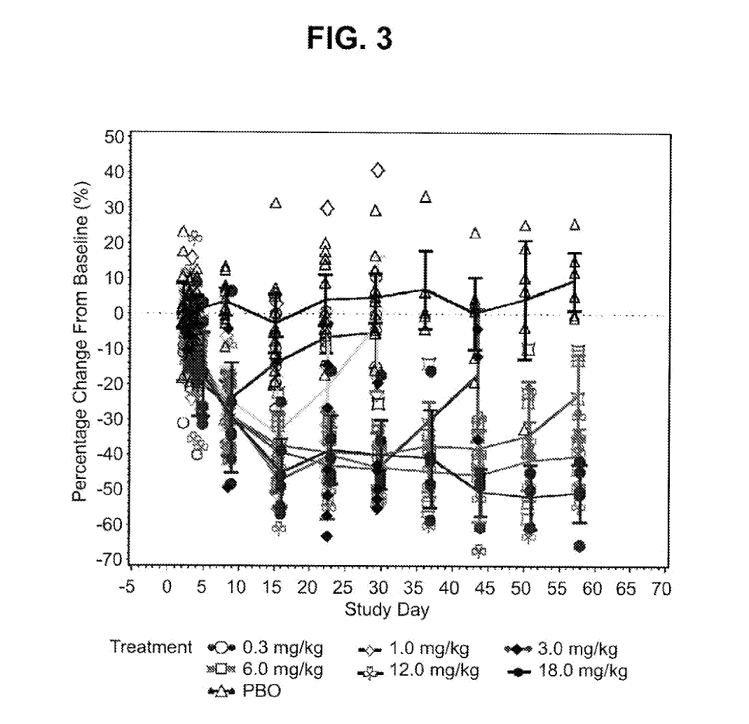

Treatment with L1L3 resulted in substantial and durable dose-dependent fasting total cholesterol (TC) lowering. The percentage change from baseline of fasting TC levels in blood is shown in FIG. 3 (data shown are mean +/−2 SE). The baseline fasting TC was about 230 mg/dL; baseline is indicated as “0” in FIG. 3. By about day 9 after dosing, TC levels in subjects dosed with a single dose of 12 or 18 mg/kg L1L3 were reduced to about 30% below baseline or lower; the TC lowering effect lasted at least to day 57 post-dosing (end of study). TC levels in subjects dosed with a single dose of 6 mg/kg L1L3 were reduced to about 30% below baseline or lower by about day 9 after dosing until about day 52 post-dosing. TC levels in subjects dosed with a single dose of 3 mg/kg L1L3 were reduced to about 30% below baseline by about day 9 after dosing, and about 40% below baseline by about day 22 after dosing. TC levels in subjects dosed with a single dose of 3 mg/kg L1L3 were reduced to about 40% below baseline by about day 22 after dosing. TC levels in subjects dosed with a single dose of 1 mg/kg L1L3 were reduced to about 36% below baseline by about day 15 after dosing. TC levels in subjects dosed with a single dose of 0.3 mg/kg L1L3 were reduced to about 25% by about day 9 after dosing. By day 15 post-dosing, a number of subjects had TC levels lower than 50% below baseline after dosing with a single dose of 12 or 18 mg/kg L1L3. By day 30 post-dosing, a number of subjects had TC levels lower than 50% below baseline after dosing with a single dose of 6 mg/kg L1L3. TC levels in subjects dosed with placebo remained at or above 2% below baseline for the duration of the study.

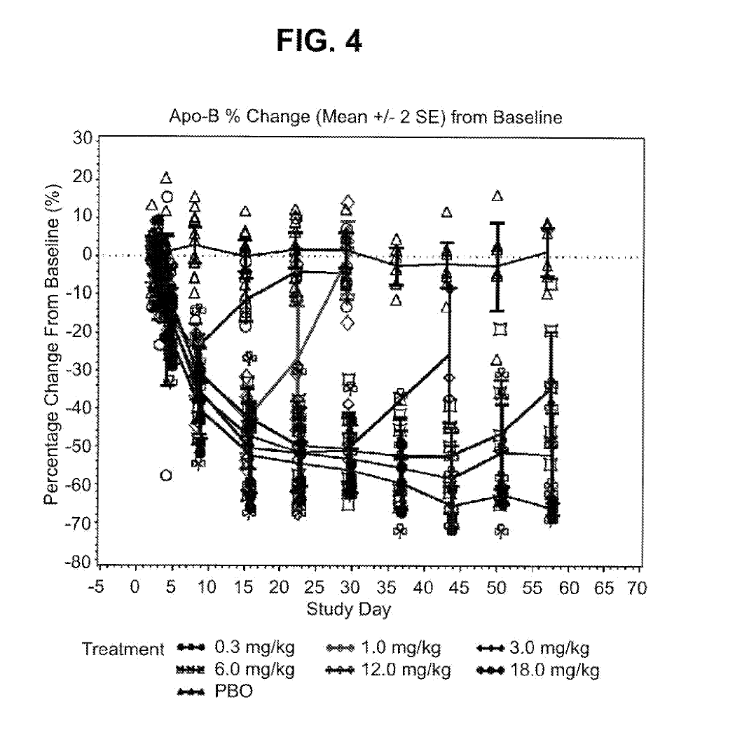

Treatment with L1L3 resulted in substantial and durable dose-dependent fasting apolipoprotein B (apo B) lowering. The percentage change from baseline of fasting apo B levels in blood is shown in FIG. 4. Data shown are mean +/−2 SE. The baseline fasting apo B level was about 119 mg/dL; baseline is indicated as “0” in FIG. 4. Apo B levels in subjects dosed with placebo remained about baseline for the duration of the study. Apo B levels in subjects dosed with 12 or 18 mg/kg L1L3 were reduced to about 50% below baseline by day 14, and remained at about 50% below baseline or lower for the remainder of the study. Apo B levels in subjects dosed with 6 mg/kg L1L3 were reduced to about 40% below baseline by day 14, about 50% below baseline by day 21, and generally below about 30% below baseline for the remainder of the study. Apo B levels in subjects dosed with 3 mg/kg L1L3 were reduced to about 40% below baseline by day 14, about 50% below baseline by day 28. Apo B levels in subjects dosed with 1 mg/kg L1L3 were reduced to about 40% below baseline by day 14. Apo B levels in subjects dosed with 0.3 mg/kg L1L3 were reduced to about 25% below baseline by day 7.

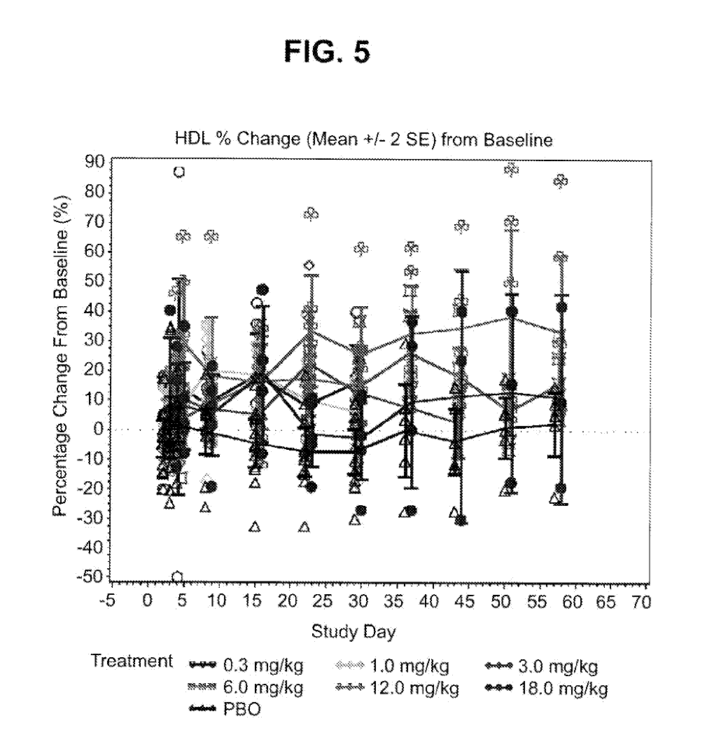

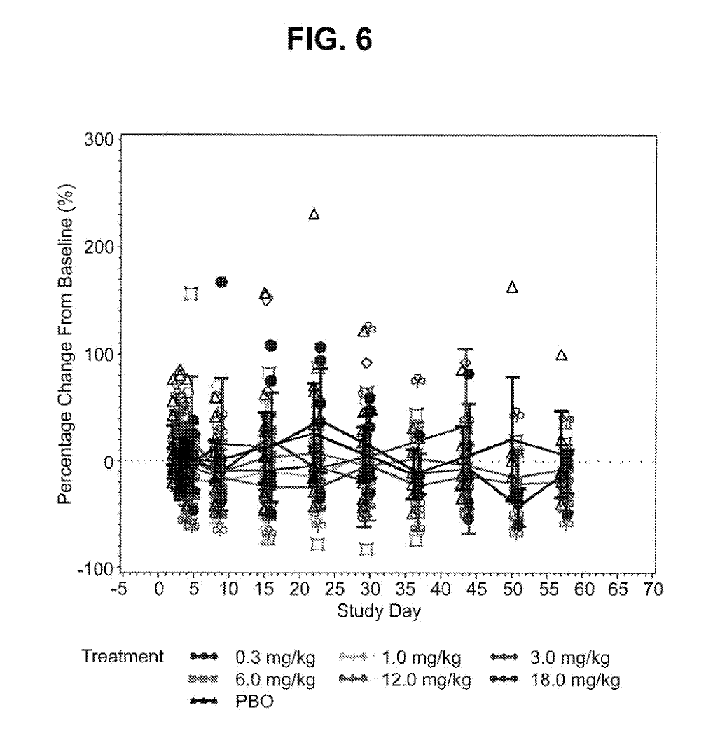

As shown in FIG. 5, high density lipoprotein cholesterol (HDL-C) levels did not change significantly after treatment with L1L3. Data shown in FIG. 5 are mean +/−2 SE. The baseline fasting HDL-C level was about 49 mg/dL; baseline is indicated as “0” in FIG. 5. HDL-C levels in subjects dosed with placebo remained about baseline for the duration of the study. Fasting triglyceride (TGs) levels remained unchanged during the study. The percentage change from baseline of fasting TG levels in blood is shown in FIG. 6. Data shown are mean +/−2 SE. The baseline fasting TG level was 173 mg/dL; baseline is indicated as “0” in FIG. 6.

In the study, no serious adverse events occurred, and there were no subjects discontinued due to treatment emergent adverse events (TEAEs). The majority of TEAEs were mild in intensity; none were severe.

In summary, administration of L1L3 resulted in a lowering of LDL-C in all dosage groups evaluated. In general, maximum percentage LDL-C lowering occurred in measurements taken on Day 15 or Day 22. The lowering effects were seen as early as Day 3. The extent and duration of LDL-C lowering was dose-dependent. The results demonstrate L1L3 has a long duration of action, i.e., with maximum effect for 7 and 14 days, for doses of 0.3 mg/kg and 1.0 mg/kg, respectively, for up to 4 weeks for a 3.0 mg/kg dose, and for more than 6 weeks, at doses of 6 mg/kg, 12 mg/kg, and 18 mg/kg L1L3 antibody. These duration effects were unexpected based upon the T_{1/2} data for L1L3.

### Example 3

### Pharmacokinetics and Pharmacodynamics of a Single dose of PCSK9 Antagonist Antibody L1L3 in Combination with Statin

This example illustrates a clinical trial study to evaluate pharmacokinetics and pharmacodynamics of a single dose of PCSK9 antagonist antibody (L1L3) in human subjects on stable doses of atorvastatin.

In the study, human subjects on stable doses of atorvastatin were administered a single dose of L1L3 antibody at either 0.5 mg/kg or 4 mg/kg of the PCSK9 antagonist antibody. L1L3 was administered as a single infusion over approximately 60 minutes. Infusion rates were carefully controlled by an infusion device per protocol. Atorvastatin 40 mg daily) was administered as described below in the study protocol. Subjects self-administered atorvastatin during their participation in this study except from Days 1 through 7 during their confinement to the clinic where the same dose was administered by qualified site personnel.

L1L3 Injection, 10 mg/mL, was presented as a sterile solution for intraveneous (IV) administration. Each vial contained 100 mg of L1L3 in 10 mL of aqueous buffered solution, and was sealed with a coated stopper and an aluminum seal. Atorvastatin (40 mg) is a white tablet coded “PD 157” on one side and “40” on the other.

Screening took place within 28 days of the dose for each subject. Subjects were on stable dosages of atorvastatin for at least 45 days prior to screening. Subjects received a single dose of L1L3 on Day 4, with multiple PK and safety assessments through the confinement period (study Days -1, 1-7). The subjects returned to the clinical research unit for subsequent visits.

Key inclusion criteria for the subjects were: on stable doses of atorvastatin (40 mg daily) for 45 days prior to Day 1, body mass index (BMI) of 18.5 to 40 kg/m2 inclusive, and body weight equal or lower than 150 kg. Key exclusion criteria for the subjects were: history of a cardiovascular event (e.g., myocardial infarction (MI)) during the past year; poorly controlled Type 1 or Type 2 Diabetes mellitus (definition: uncontrolled diabetes is defined as HBIAc >9%); and poorly controlled hypertension (uncontrolled hypertension is defined as a systolic blood pressure greater than 140 mm Hg or a diastolic blood pressure greater than 90 mm Hg, even with treatment). Subjects who have hypertension and are controlled on stable dosages of anti-hypertensive medications could be included. The study included both genders, with a minimum age limit of 18 and a maximum age limit of 80.

Pharmacokinetics parameter estimates of L1L3 antibody in the presence of atorvastatin and of atorvastatin were evaluated after a single dose of 0.5 or 4 mg/kg

L1L3 antibody. The absolute and percent change from baseline of fasting LDL cholesterol (LDL-C) were measured after L1L3 antibody administration. In the study, the incidence of subjects meeting toxicity or intolerable dose criteria was measured. Incidence of treatment emergent adverse events (TEAEs) categorized by severity and causal relationship to study drug was also be measured. The timeframe for measurement of each of the above outcomes was two months.

Study Protocol

Day-1: Subjects were admitted to the clinical research unit (CRU), and the following were completed: reviewed and update inclusion and exclusion criteria; reviewed and update medical history; reviewed and update history of all prescription or nonprescription drugs, and dietary supplements taken within 28 days prior to the planned first dose; brief physical examination; vitals sign measurements (blood pressure, pulse rate, body temperature) supine and standing; collected blood and urine specimens for safety laboratory tests (serum chemistry; hematology, urinalysis, coagulation, lipase, amylase, CRP) following a 10-hour fast; urine drug and alcohol screen test; urine pregnancy test (females of childbearing potential); collected blood sample for immunogenicity analysis (Anti-L1L3 Antibody); collected blood sample for pharmacodynamic analysis (PCSK9 and Lipid Particle); collected blood sample for pharmacogenomics (optional, subject's consent required); triplicate, supine ECG; assessed alcohol, caffeine and tobacco use; assessed baseline symptoms/adverse events; and randomized subject.

Day 1: Prior to dosing, the following were completed: triplicate, supine ECG (prior to inserting IV catheter, if applicable); vital signs measurements (blood pressure, pulse rate, body temperature) supine and standing; collected (Day 1, 0 hr.) blood sample for PK (atorvastatin); subjects took the sponsor-provided atorvastatin dose (40 mg); post dosing, blood samples for PK (atorvastatin) were collected at the following time points for Day 1: .25, .5, 1, 2, 3, 4, 6, 8 and 12 hours. The following were completed: assessed baseline symptoms/adverse events; reviewed concomitant medications. Subjects fasted at least 10 hours prior to the lipid panel blood sample on Day 2.

Day 2: Prior to dosing, the following were completed: vital signs measurements (blood pressure, pulse rate, body temperature) supine and standing; collected (Day 2, 0 hr) blood sample for PK (atorvastatin); collected lipid panel following a 10-hour fast; subjects took the sponsor-provided atorvastatin dose (40 mg). The following were completed: assessed baseline symptoms/adverse events and reviewed concomitant medications.

Day 3: Prior to dosing, the following were completed: collected Day 3, 0 hr) blood sample for PK (atorvastatin); vitals signs measurements (blood pressure, pulse rate, body temperature) supine and standing; subjects took the sponsor-provided atorvastatin dose (40 mg). The following were completed: assessed baseline symptoms/adverse events; reviewed concomitant medications. Subjects fasted at least 10 hours prior to the lipid panel blood sample on Day 4.

Day 4: Prior to dosing with atorvastatin and L1L3, the following were completed: triplicate, supine ECG; vital signs measurements (blood pressure, pulse rate, body temperature) supine and standing; collected (Day 4, 0 hr.) blood samples for atorvastatin PK; collected (Day 4, 0 hr) blood samples for L1L3 PK; collected blood and urine specimens for safety laboratory tests (serum chemistry; hematology, urinalysis, lipase, amylase, CRP) following a 10-hour fast; weight; collected lipid panel following a 10-hour fast; collected blood sample for pharmacodynamic analyses (PCSK9 and Lipid Particle); collected blood sample for immunogenicity (Anti-L1L3 Antibodies). Dose Administration: subjects took sponsor-provided atorvastatin (40 mg). L1L3 was administered by rate controlled intravenous infusion over approximately 60 minutes. Post dose administrations, the following were completed: collected blood samples for PK (atorvastatin) for Day 4 at .25, .5, 1, 2, 3, 4, 6, 8, and 12 hours post atorvastatin dose; collected blood samples for PK (L1L3) for Day 4 at 1, 4, 8, and 12 hours from start of infusion; triplicate, supine ECG 1 hour post dose; vital signs measurements (blood pressure, pulse rate, body temperature) supine and standing at 1 and 4 hours from start of the L1L3 infusion; and assessed baseline symptoms/adverse events; reviewed concomitant medications. Subjects fasted at least 10 hours prior to the lipid panel blood sample on Days 5 and 6.

Days 5 and 6: Prior to dosing, the following were completed: vital signs measurements (blood pressure, pulse rate, body temperature) supine and standing; collected (Day 5, 0 hr.) blood sample for PK (atorvastatin); collected (Day 5) blood sample for PK (L1L3) ; collected lipid panel following a 10-hour fast. Day 5 only: collected blood sample for pharmacodynamic analyses (PCSK9 and Lipid Particle).

Subjects took the sponsor-provided atorvastatin dose (40 mg). The following were completed: assessed baseline symptoms/adverse events; reviewed concomitant medications. Subjects fasted at least 10 hours prior to the lipid panel blood sample on Day 7.

Day 7: Prior to dosing, the following were completed: triplicate, supine ECG;

vitals sign measurements (blood pressure, pulse rate, body temperature) supine and standing; collected (Day 7) blood sample for PK (atorvastatin); collected (Day 7) blood sample for PK (L1L3); collected lipid panel following a 10-hour fast; collected blood sample for pharmacodynamic analysis (PCSK9 and Lipid Particle); collected blood and urine specimens for safety laboratory tests (serum chemistry; hematology, urinalysis, coagulation, lipase, amylase, CRP) following a 10-hour fast. Subjects took the last sponsor-provided atorvastatin dose (40 mg). Prior to discharge from the unit, the following were completed: brief physical examination; assessed baseline symptoms/adverse events; reviewed concomitant medications. Subjects were reminded to return to the clinic and to fast at least 10 hours prior to the lipid panel blood sample on Day 15. Subjects continued taking their prescribed atorvastatin medication throughout the remainder of the study.

Day 15 (±1 day): The following were completed: brief physical examination; compliance check for atorvastatin; standard, supine ECG; vitals sign measurements (blood pressure, pulse rate, body temperature) supine and standing; collected (Day 15) blood sample for PK (L1L3); collected lipid panel following a 10-hour fast; collected blood sample for immunogenicity (Anti-L1L3 Antibodies) ; collected blood sample for pharmacodynamic analysis (PCSK9 and Lipid Particle); collected blood and urine specimens for safety laboratory tests (serum chemistry, hematology, urinalysis, CRP) following a 10-hour fast; assessed baseline symptoms/adverse events; reviewed concomitant medications. Subjects were reminded to return to the clinic and to fast at least 10 hours prior to the lipid panel blood sample on Day 22.

Day 22 (±1 day): The following were completed: brief physical examination; compliance check for atorvastatin; vitals sign measurements (blood pressure, pulse rate, body temperature) supine and standing; collected (Day 22) blood sample for PK (L1L3); collected lipid panel following a 10-hour fast; collected blood and urine specimens for safety laboratory tests (serum chemistry, hematology, urinalysis, CRP) following a 10-hour fast; assessed baseline symptoms/adverse events; reviewed concomitant medications. Subjects were reminded to return to the clinic and to fast at least 10 hours prior to the lipid panel blood sample on Day 29.

Day 29 (±1 day): The following were completed: complete physical examination; compliance check for atorvastatin; vitals sign measurements (blood pressure, pulse rate, body temperature) supine and standing; collected (Day 29) blood sample for PK (L1L3); collected blood sample for pharmacodynamic analyses (PCSK9 and Lipid Particle); collected blood sample for immunogenicity (Anti-L1L3 Antibodies); collected lipid panel following a 10-hour fast; triplicate, supine ECG; collected blood and urine specimens for safety laboratory tests (serum chemistry, hematology, urinalysis, coagulation, lipase, amylase) following a 10-hour fast, urine drug and alcohol screen test; serum pregnancy test (females of childbearing potential); assessed baseline symptoms/adverse events; reviewed concomitant medications. Subjects were reminded to return to the clinic and to fast at least 10 hours prior to the lipid panel blood sample on Day 36.

Days 36, 43, 50, 57, and 64 (Termination Visit): The following were completed: brief physical examination; compliance check for atorvastatin; standard, supine ECG; vitals sign measurements (blood pressure, pulse rate, body temperature) supine and standing; collected blood sample for PK (L1L3); collected blood sample for immunogenicity (Anti-L1L3 Antibodies); collected lipid panel following a 10-hour fast; collected blood and urine specimens for safety laboratory tests (serum chemistry, hematology, urinalysis, lipase, amylase, CRP) following a 10-hour fast; assessed baseline symptoms/adverse events; reviewed concomitant medications. Day 64 Only: urine pregnancy test (females of childbearing potential); coagulation Panel; weight; collected blood sample for pharmacodynamic analyses (PCSK9 and Lipid Particle).

Day 78 and 92: In some instances, two visits were added, Day 78 and 92, pending the pharmacokinetic results from Day 57. In this event, the procedures for Day 57 were followed for Day 78, and the procedures for Day 64 were followed for Day 92. Day 92 became the termination visit.

Results

There were no discontinued subjects in the study. There was one serious adverse event (SAE), i.e. worsening of migraine headache, which was not drug-related. The TEAEs were generally nonspecific, and none were severe in intensity. In addition, the TEAEs were transient, with greater than 3× ULN alanine aminotransferase (ALT) and/or aspartate aminotransferase (AST), without clinical signs/symptoms, and all were resolved within one week.

Table 4 summarizes the L1L3 PK parameters of this study.

TABLE 4
L1L3 PK Parameters: Geometric Mean (CV %)

| | 4 mg/kg L1L3 + | 0.5 mg/kg L1L3 + |
|---|---|---|
| Parameter | Atorvastatin | Atorvastatin |
| N, n | 12,12 | 7,7 |
| AUC,_{inf} (ng · day/mL) | 777167 (13) | 46338 (28) |
| AUCₜ (ng · day/mL) | 726337 (17) | 38310 (35) |
| Cₘₐₓ (ng/mL) | 105048 (16) | 13827 (9) |
| Tₘₐₓ (day) | 0.1 (0.04-0.50) | 0.17 (0.04-0.33) |
| t_{1/2} (day) | 7.3 (33) | 2.6 (34) |
| CL (mL/day/kg) | 5.2 (15) | 10.8 (29) |
| Vss (mL/kg) | 52.3 (16) | 40.2 (14) |

Table 5 summarizes the results from this clinical trial study to evaluate pharmacokinetics and pharmacodynamics of a single dose of L1L3 in human subjects on stable doses of atorvastatin. The mean percent change from baseline of fasting LDL-C levels after L1L3 antibody administration is provided (Table 4).

TABLE 5
Mean (SD) LDL-C vs Time Data

| | 0.5 mg/kg L1L3 | | 4 mg/kg L1L3 | |
|---|---|---|---|---|
| | (n = 12) | | (n = 12) | |
| Day | Mean | SD | Mean | SD |
| 0 | 0.0 | 0.0 | 0.0 | 0.0 |
| 1 | −28.8 | 20.2 | −20.9 | 18.5 |
| 2 | −48.5 | 26.3 | −38.4 | 13.0 |
| 3 | −66.7 | 28.2 | −43.3 | 18.1 |
| 11 | −34.4 | 27.0 | −64.6 | 26.0 |
| 18 | 9.0 | 39.7 | −73.2 | 21.2 |
| 25 | 23.3 | 43.5 | −70.8 | 20.4 |
| 32 | 14.8 | 37.0 | −69.9 | 14.8 |
| 39 | 21.2 | 36.7 | −45.1 | 16.9 |
| 46 | 17.0 | 37.2 | −19.2 | 16.4 |
| 53 | 27.9 | 42.7 | −3.6 | 25.4 |
| 60 | 30.1 | 39.3 | 7.1 | 25.8 |

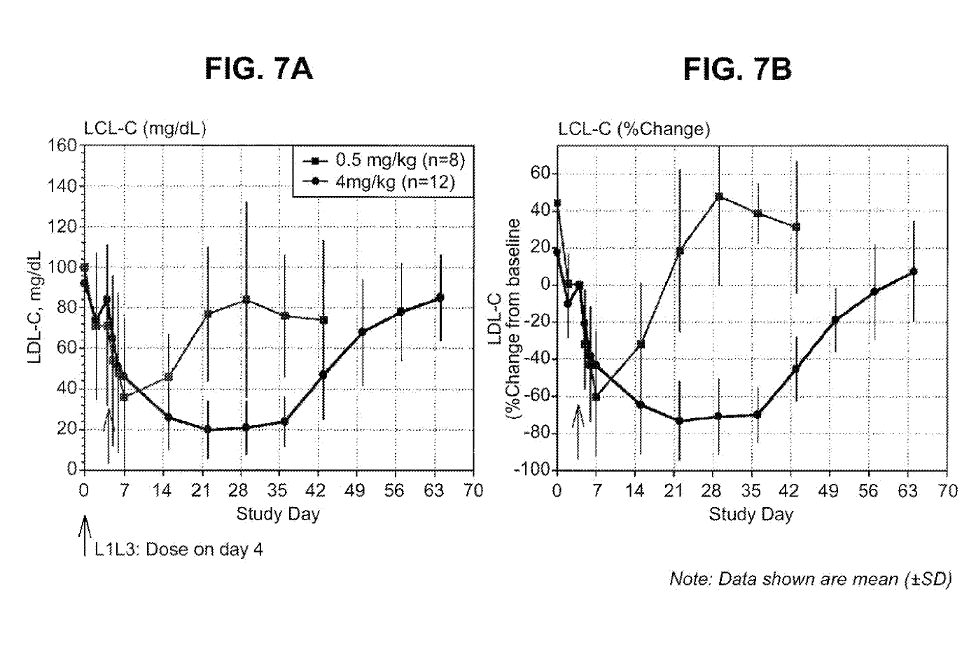

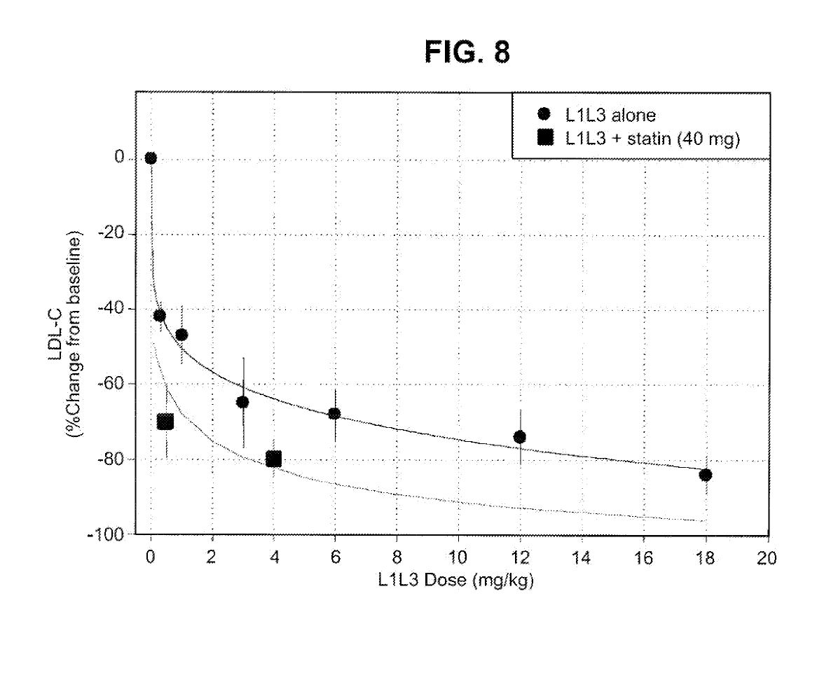

Treatment with L1L3 in the presence of atorvastatin (dose=40 mg) resulted in substantial and durable dose-dependent fasting LDL-C lowering. The baseline fasting LDL C was about 72.5 mg/dL. FIG. 7A depicts absolute fasting LDL-C levels after L1L3 antibody administration. FIG. 7B depicts the percent change from baseline of fasting LDL-C levels after L1L3 antibody administration. Baseline is indicated as “0” in FIG. 7B. With an L1L3 dose of 0.5 mg/kg, the maximum LDL-C lowering effect was observed on day 3 following L1L3 administration. With an L1L3 dose of 4 mg/kg, the maximum LDL-C lowering effect was observed through day 32 following L1L3 administration. The dose-dependent response in LDL-C lowering is shown in FIG. 8. As shown in FIG. 8, L1 L3 lowered LDL-C in patients on stable doses of statin at every dose administered. Furthermore, the LDL-C lowering effect in patients on stable doses of statin was greater than the effect in patients dosed with L1L3 alone (FIG. 8).

### Example 4

### PK-PD Modeling and Simulated Time Profiles

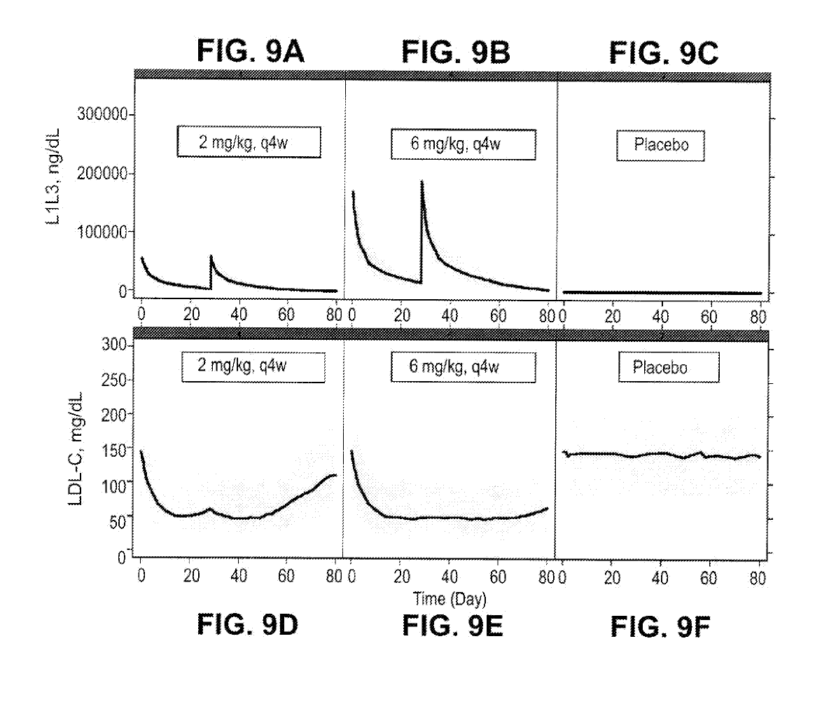

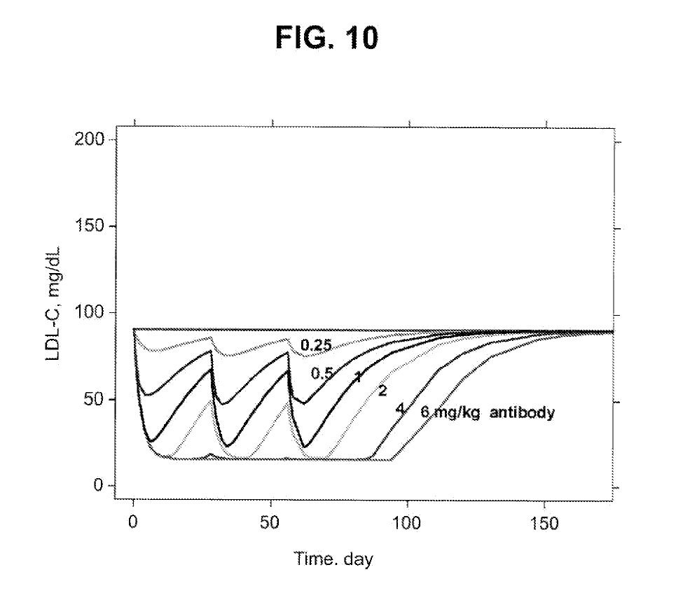

Based on the data provided in the studies described above, simulated serum L1L3-time profiles and LDL-C-time profiles were generated. FIGS. 9A-F depict graphs of simulated time profiles for L1L3 (top panel) and LDL-C (bottom panel) after administration of L1L3 at the indicated doses, or placebo. The simulated profiles were generated for dosing with 2 mg/kg L1L3 (left) or 6 mg/kg L1L3 (middle) compared to placebo (right). L1L3 or placebo was administered at Day 0 and Day 29, i.e., two doses four weeks apart. FIG. 10 depicts the simulated LDL-C-time profiles after administration of following L1L3 dose amounts: 0.25 mg/kg, 0.5 mg/kg, 1 mg/kg, 2 mg/kg, 4 mg/kg and 6 mg/kg, each administered at Day 0, Day 29 and Day 56 (FIG. 10). The simulated L1L3-time profiles and LDL-C-time profiles demonstrate that low doses of L1L3 administered once every four weeks produces sustained LDL-C lowering.

### Example 5

### Pharmacokinetics and Pharmacodynamics Following Multiple doses of L1L3

This example illustrates a clinical trial study to evaluate pharmacokinetics and pharmacodynamics following multiple intravenous doses of PCSK9 antagonist antibody (L1L3) in human subjects.

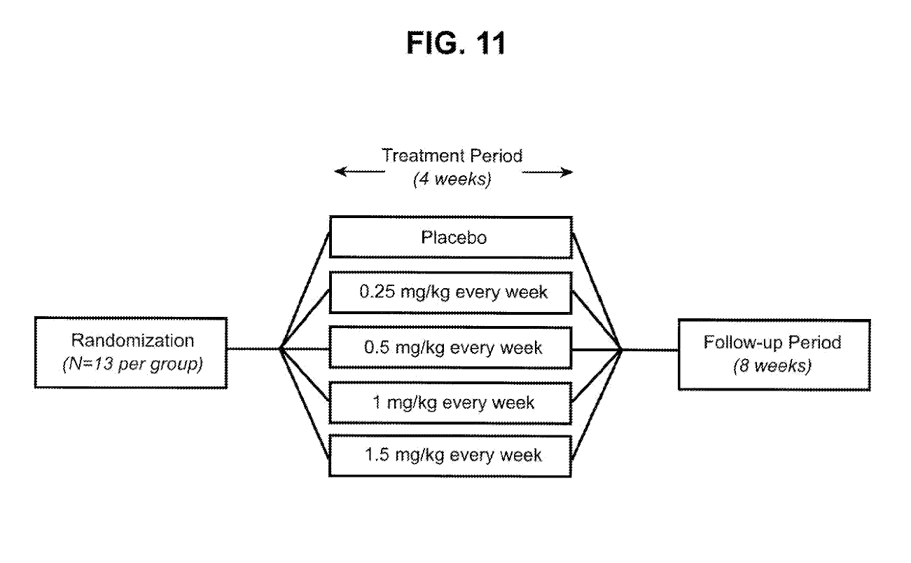

This study was a randomized, multi-center, double-blind, placebo control, parallel designed trial with a 28 day screening period, 4 week treatment period and 8 week follow-up period (FIG. 11). In the study, human Japanese subjects were administered L1L3 antibody at 0.25 mg/kg, 0.5 mg/kg, 1.0 mg/kg, or 1.5 mg/kg of the PCSK9 antagonist antibody. For each subject, the study consisted of 3 periods: screening, treatment, and follow-up. The treatment period lasted up to approximately 28 days with 4 single I.V. doses of either L1L3 or placebo administered on Days 1, 8, 15, and 22. The follow-up period will lasted approximately 8 weeks, from approximately Day 29 to the last visit (Day 78). Subjects were seen periodically in the clinic for safety assessments and collection of blood for routine laboratory tests, lipid profiles, PK, PD, and immunogenicity samples.

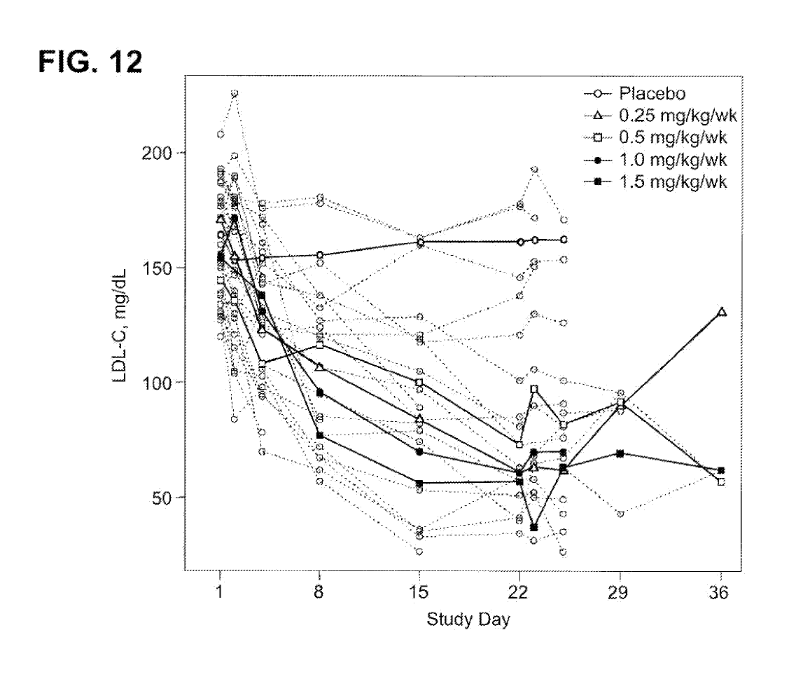

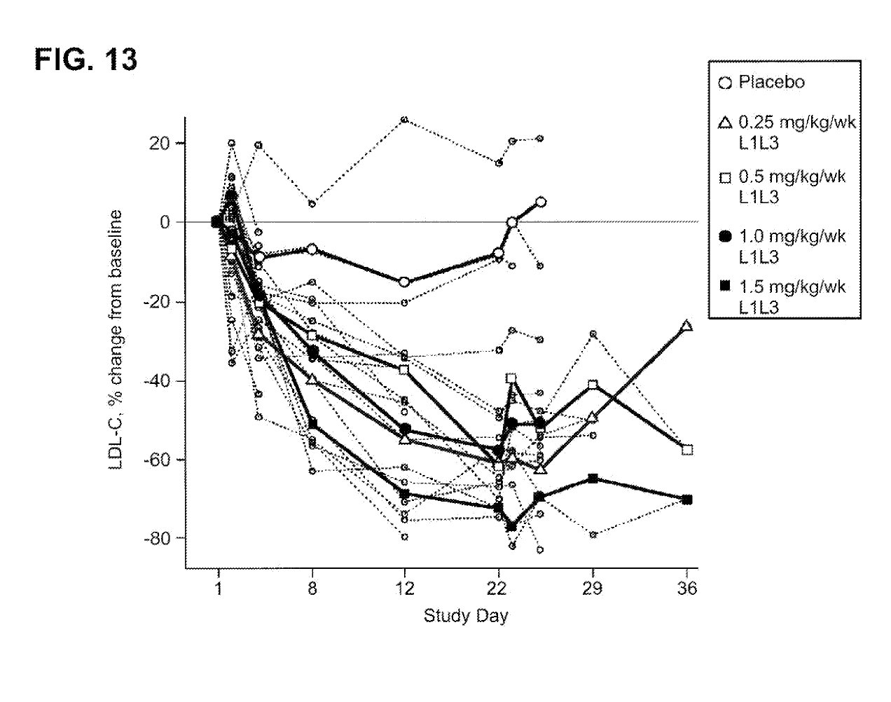

Weekly treatment with L1L3 at all doses tested resulted in sustained, substantial and durable dose-dependent fasting LDL-C lowering. The baseline fasting LDL-C was about 155 mg/dL. FIG. 12 depicts absolute fasting LDL-C levels after L1L3 antibody administration. FIG. 13 depicts the percent change from baseline of fasting LDL-C levels after L1L3 antibody administration. Baseline is indicated as “0” in FIG. 13.

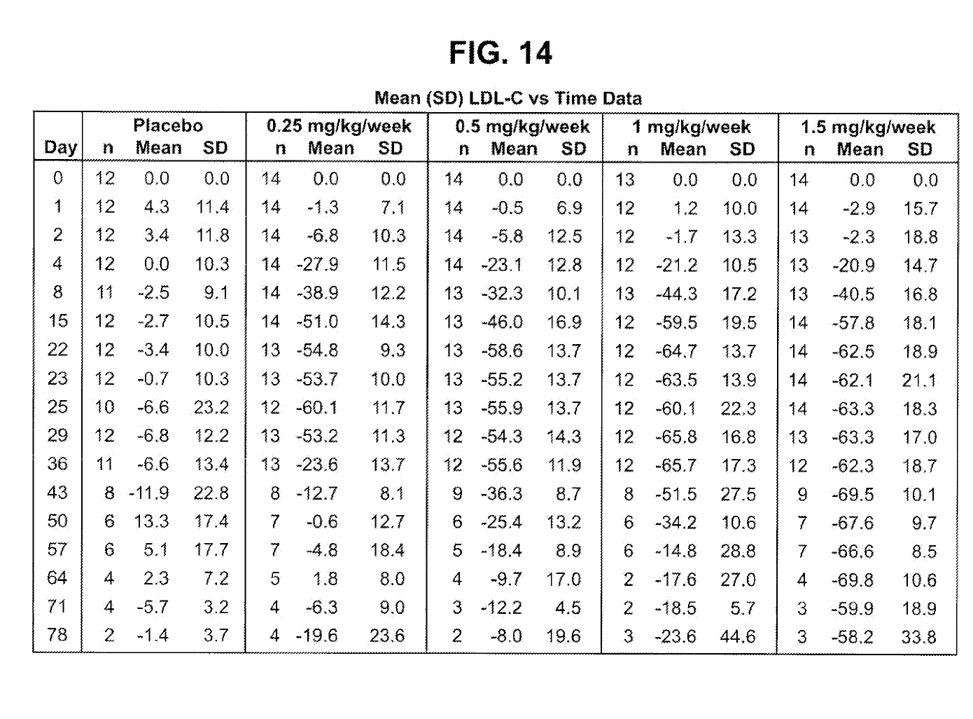

The table in FIG. 14 summarizes the results from this clinical trial study to evaluate pharmacokinetics and pharmacodynamics following multiple doses of L1L3 in human subjects on stable doses of atorvastatin. The mean percent change from baseline of fasting LDL-C levels after L1L3 antibody administration is provided (“Mean”) (FIG. 14).

### Example 6

### Pharmacokinetics and Pharmacodynamics Following Multiple doses of L1L3 in Combination with Statin

This example illustrates a clinical trial study to evaluate pharmacokinetics and pharmacodynamics following multiple intravenous doses of PCSK9 antagonist antibody (L1L3) in human subjects on atorvastatin, simvastatin or rosuvastatin.

This study was a randomized, multi center, double blind, placebo control, parallel designed trial with a 3 week screening period, 12 week treatment period and 8 week follow up period.

Subjects enrolled in the study met all of the following criteria: men and women subjects greater than equal to age of 18; body mass index of 18.5 to 40 kg/m²; total body weight greater than 50 kg (110 lbs) and less than 150 kg (330 lbs); on a stable daily dose of a statin, defined as atorvastatin 40 or 80 mg, rosuvastatin 20 or 40 mg or simvastatin 40 or 80 mg for a minimum of 45 days prior to Day 1; lipids meet the following criteria at two qualifying visits (screening and Day-7): fasting LDL-C greater than or equal to 100 mg/dL, ;

Subjects were seen periodically in the clinic for safety assessments and collection of blood for safety labs, lipid profiles, PK, PD, and immunogenicity samples. Telephone contacts were made prior to each visit to remind them of the 10-hour fasting requirements, during screening and on Day 3 to assess adverse events and document the contact in the subject's source document. Subjects received one infusion of 1 mg/kg L1L3, 3 mg/kg L1L3, 6 mg/kg L1L3, or placebo on Days 1, 29 and 57 with multiple efficacy, safety and PK assessments throughout the treatment and follow-up periods. Infusion rates were carefully controlled by an infusion device per protocol. Infusions were administered as a single infusion over approximately 60 minutes.

Results

The 3 mg/kg dosage regimen and 6 mg/kg dosage regimen both achieved statistical significance and exceeded the target value of 30% change in LDL-C from baseline. No effect of L1L3 on triglycerides was observed. A slight elevation of HDL up to 9% was seen. The treatment groups and enrollment are shown in Table 6.

TABLE 6

| | | L1L3 | | | |
|---|---|---|---|---|---|
| | | 0.25 | L1L3 | L1L3 | L1L3 |
| | Placebo | mg/kg | 1 mg/kg | 3 mg/kg | 6 mg/kg |
| | (N = 19) | (N = 19) | (N = 18) | (N = 18) | (N = 18) |
| #of Subjects: | n (%) | n (%) | n (%) | n (%) | n (%) |
| Atorvastatin | 6 (31.6) | 6 (31.6) | 6 (33.3) | 6 (33.3) | 6 (33.3) |
| Rosuvastatin | 6 (31.6) | 5 (26.3) | 6 (33.3) | 5 (27.8) | 5 (27.8) |
| Simvastatin | 7 (36.8) | 6 (31.6) | 6 (33.3) | 7 (38.9) | 6 (33.3) |

The pre-specified primary efficacy endpoint was the percentage change from baseline of LDL-C at Day 85 analyzed using an ANCOVA model. The final ANCOVA model contained terms for baseline LDL-C and treatment. To preserve the overall type I error rate at a level of 0.05 for the primary endpoint analysis, a Haybittle-Peto, boundary with 0.001 alpha spent was employed.

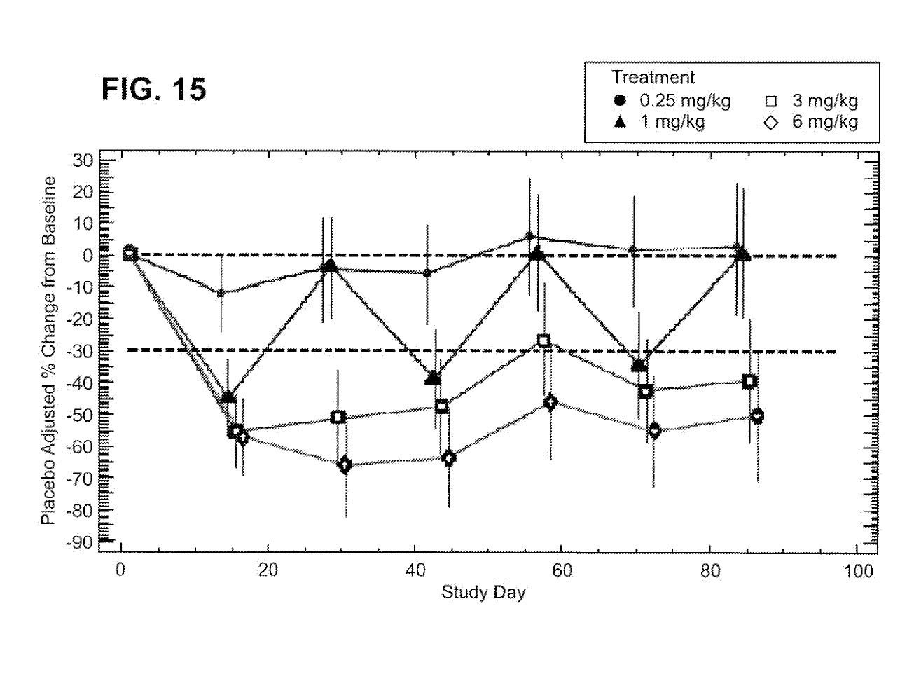

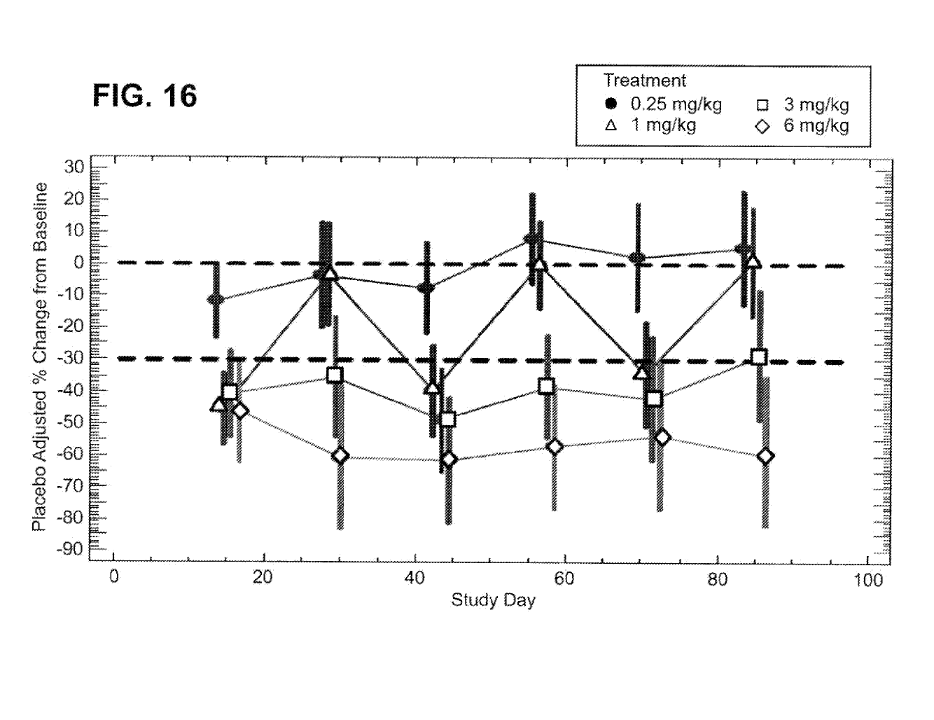

A strong treatment effect with a clear dose response was observed with variation in LDL-C for the 3 and 6 mg/kg treatment groups driven by the missing-ness of doses (FIGS. 15 and 16). The LDL-C data were subsequently analyzed using mixed model repeated measures to estimate both the treatment by time and empirical dose-response profiles.

The pre-determined target value of additional 30% LDL-C when added to statins was the proof-of-concept criterion of success. This target level of 30% of LDL-C lowering or more, when added to statin therapy, was clearly achieved with the 3 and 6 mg/kg doses given every 4 weeks (FIGS. 15 and 16). The graph in FIG. 15 shows the percent change from baseline by study day and treatment, and the graph in FIG. 16 shows the percent change from baseline by study day and treatment excluding the subjects with missed doses. The 3 mg/kg L1L3 dosing regimen in patients on a stable daily dose of a statin achieved LDL-C lowering to about 50% below baseline by Day 29 (FIG. 15). The 6 mg/kg L1L3 dosing regimen in patients on a stable daily dose of a statin achieved LDL-C lowering to about 65% below baseline by Day 29 (FIG. 15). With both the 3 mg/kg and 6 mg/kg dosing regimens, greater than 30% LDL-C lowering persisted for 28 days (FIG. 16). A statistical summary of the placebo adjusted treatment effects at Day 85 is provided in Table 7. In Table 7, the baseline of lipid profile is defined as the average of values observed at Days -7 and 1.

TABLE 7
Summary of Statistical Analysis (MMRM) of Percentage Changes from Baseline for
LDL-C Data on Day 85

| | Difference | | | |
|---|---|---|---|---|
| Comparison | in LS means | Standard | | |
| (Test vs. Reference) | (Test-Reference) | Error | 95% CI | *P-value |
| L1L3 0.25 mg/kg vs. Placebo on Day 85 | 2.67 | 10.252 | (−17.87, 23.20) | 0.7958 |
| L1L3 1 mg/kg vs. Placebo on Day 85 | 0.83 | 10.013 | (−19.23, 20.89) | 0.9340 |
| L1L3 3 mg/kg vs. Placebo on Day 85 | −38.92 | 9.721 | (−58.39, −19.46) | 0.0002 |
| L1L3 6 mg/kg vs. Placebo on Day 85 | −50.14 | 10.266 | (−70.70, −29.57) | <0.0001 |

A summary of L1L3 Cmax and trough concentrations is shown in Table 8.

TABLE 8
L1L3 Pharmacokinetics

| Dosage | After 1^{st} Dose | | After 3^{rd} Dose | |
|---|---|---|---|---|
| (mg/kg) | Cₘₐₓ (μg/mL) | C_{trough}(μg/mL) | Cₘₐₓ (μg/mL) | C_{trough}(μg/mL) |
| 0.25 | 10.9 ± 13.0 | 0.109 ± 0.406 | 6.95 ± 1.55 | 0.122 ± 0.226 |
| | (n = 17) | (n = 14) | (n = 13) | (n = 8) |
| 1 | 28.3 ± 6.6 | 0.256 ± 0.310 | 37.3 ± 26.4 | 0 ± 0 |
| | (n = 17) | (n = 14) | (n = 11) | (n = 9) |
| 3 | 92.2 ± 22.6 | 3.16 ± 2.30 | 86.5 ± 15.0 | 3.04 ± 4.42 |
| | (n = 18) | (n = 13) | (n = 12) | (n = 7) |
| 6 | 182 ± 64 | 17.4 ± 11.6 | 179 ± 66 | 15.6 ± 15.3 |
| | (n = 17) | (n = 14) | (n = 8) | (n = 7) |

Monthly treatment with L1L3 at 3 and 6 mg/kg in patients on a stable daily dose of a statin resulted in greater than 30% lowering of blood LDL-C levels from baseline. Minor elevations (up to 9%) in HDL levels and little effects of L1L3 on triglycerides were observes. L1L3 was generally safe and well-tolerated. Changes in LFTs, CK, ECGs, and BP were transient, mild in nature and in most cases were considered not related to treatment. No subject had positive ADA.

### Example 7

### Pharmacokinetics and Pharmacodynamics Following Multiple doses of L1L3 in Combination with Statin

This example illustrates a clinical trial study to evaluate LDL-C levels following multiple subcutaneous doses of PCSK9 antagonist antibody (L1L3) in human subjects on a statin.

This study is a randomized, multi center, double blind, placebo control, parallel group, dose-ranging study designed trial to assess the efficacy, safety and tolerability of L1L3 following monthly and twice monthly subcutaneous dosing for six months in hypercholesterolemic subjects on a statin. A total of 7 dose groups in two dosing schedules (Q28d or Q14d), with 50 subjects per dose group are planned. Protocol design is set forth in Table 9.

TABLE 9

| Arms | Assigned Interventions |
|---|---|
| Experimental: Q28d Dosing Arm | Group 1: Placebo, Q28d |
| Q28d dose groups will receive | Group 2: L1L3 200 mg, Q28d |
| subcutaneous administration | Group 3: L1L3 300 mg, Q28d |
| of L1L3 antibody | |
| or Placebo once a month. | |
| Experimental: Q14d Dosing | Group 4: Placebo, Q14d |
| Arm Q14d dose groups | Group 5: L1L3 50mg, Q14d |
| will receive subcutaneous | Group 6: L1L3 100 mg, Q14d |
| administration of L1L3 | Group 7: L1L3 150 mg, Q14d |
| antibody or Placebo every 2 weeks. | |

Eligibility: ages 18 years or older.

Inclusion criteria: subjects should be receiving stable doses (at least 6 weeks) of any statin and continue on same dose of statin for the duration of this trial. Lipids should meet the following criteria on a background treatment with a statin at 2 screening visits that occur at screening and at least 7 days prior to randomization on Day 1: fasting LDL-C greater than or equal to 80 mg/dL (2.31 mmol/L); fasting TG less than or equal to 400 mg/dL (4.52 mmol/L); subject's fasting LDL-C must be greater than or equal to 80 mg/dL (2.31 mmol/L at the initial screen visit, and the value at the second visit within 7 days of randomization must be not lower than 20% of this initial value to meet eligibility criteria for this trial.

The primary outcome measure will be the absolute change from baseline in LDL-C at the end of week 12 following randomization. Secondary outcome measures include the following: LDL-C will be assessed as change and % change from baseline at the end of week 12 following randomization; plasma steady-state L1L3 pharmacokinetic parameters; proportion of subjects having LDL-C less than specified limits (<100 mg/dL, <70 mg/dL, <40 mg/dL, <25 mg/dL); total cholesterol will be assessed as change and % change from baseline at the end of week 12 following randomization; ApoB will be assessed as change and % change from baseline at the end of week 12 following randomization; ApoA1 will be assessed as change and % change from baseline at the end of week 12 following randomization; lipoprotein (a) will be assessed as change and % change from baseline at the end of week 12 following randomization; HDL-cholesterol will be assessed as change and % change from baseline at the end of week 12 following randomization; very low density lipoprotein-cholesterol will be assessed as change and % change from baseline at the end of week 12 following randomization; triglycerides will be assessed as change and % change from baseline at the end of week 12 following randomization; and non-HDL-cholesterol will be assessed as change and % change from baseline at the end of week 12 following randomization.

Although the disclosed teachings have been described with reference to various applications, methods, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1-13. (canceled)

14. A method for the treatment of a patient susceptible to or diagnosed with a disorder characterized by an elevated low-density lipoprotein cholesterol (LDL-C) level in the blood, comprising:
administering to the patient an initial dose of at least about 3 mg/kg, about 4 mg/kg, about 5 mg/kg, or about 6 mg/kg of a proprotein convertase subtilisin kexin type 9 (PCSK9) antagonist antibody; and
administering to the patient a plurality of subsequent doses of the antibody in an amount that is about the same as or less than the initial dose, wherein the initial dose and the first subsequent and additional subsequent doses are separated in time from each other by at least about four weeks.

15. A method for the treatment of a patient susceptible to or diagnosed with a disorder characterized by an elevated low-density lipoprotein cholesterol (LDL-C) level in the blood, comprising:
administering to the patient an initial dose of at least about 200 mg or about 300 mg of a proprotein convertase subtilisin kexin type 9 (PCSK9) antagonist antibody; and
administering to the patient a plurality of subsequent doses of the antibody in an amount that is about the same as or less than the initial dose, wherein the initial dose and the first subsequent and additional subsequent doses are separated in time from each other by at least about four weeks.

16. A method for the treatment of a patient susceptible to or diagnosed with a disorder characterized by an elevated low-density lipoprotein cholesterol (LDL-C) level in the blood, comprising:
administering to the patient an initial dose of at least about 50 mg, about 75 mg, about 100 mg, about 140 mg, or about 150 mg of a proprotein convertase subtilisin kexin type 9 (PCSK9) antagonist antibody; and
administering to the patient a plurality of subsequent doses of the antibody in an amount that is about the same as or less than the initial dose, wherein the initial dose and the first subsequent and additional subsequent doses are separated in time from each other by at least about two weeks.

17. The method of claim 16, wherein the patient is being treated with a statin.

18. The method of claim 17, wherein the patient is being treated with a daily dose of a statin.

19. The method of claim 17, wherein the patient has been receiving stable doses of the statin for at least about two, three, four, five or six weeks prior to the initial dose of PCSK9 antibody.

20. The method of claim 17, wherein the statin is atorvastatin, cerivastatin, fluvastatin, lovastatin, mevastatin, pitavastatin, pravastatin, rosuvastatin, simvastatin, or any pharmaceutically acceptable salts, or stereoisomers, thereof.

21. The method of claim 18, wherein the daily statin dose is selected from the group consisting of 40 mg atorvastatin, 80 mg atorvastatin, 20 mg rosuvastatin, 40 mg rosuvastatin, 40 mg simvastatin, and 80 mg simvastatin.

22. The method of claim 16, wherein the disorder is hypercholesterolemia, dyslipidemia, hyperlipidemia, atherosclerosis, cardiovascular disease, or acute coronary syndrome (ACS).

23. The method of claim 16, wherein the patient has a fasting total cholesterol level of about 70 mg/dL or greater prior to administration of the initial dose of PCSK9 antagonist antibody.

24. The method of claim 16, wherein the patient has a fasting LDL cholesterol level of about 130 mg/dL or greater prior to administration of the initial dose of PCSK9 antagonist antibody.

25. The method of claim 16, wherein the antibody comprises three CDRs from a heavy chain variable region having the amino acid sequence shown in SEQ ID NO: 11 and three CDRs from a light chain variable region having the amino acid sequence shown in SEQ ID NO: 12.

26. The method of claim 25, wherein the antibody is L1L3.

27. The method of claim 16, wherein the antibody is administered subcutaneously or intravenously.

28. The method of claim 16, wherein the antibody is administered about once or twice a month.

29. An article of manufacture, comprising a container, a composition within the container comprising a PCSK9 antagonist antibody, and a package insert containing instructions to administer an initial dose of PCSK9 antagonist antibody of at least about 3 mg/kg, about 6 mg/kg, about 50 mg, about 75 mg, about 100 mg, about 140 mg, about 150 mg, about 200 mg, or about 300 mg, and at least one subsequent dose that is the same amount or less than the initial dose, wherein administration of the initial dose and subsequent doses are separated in time by at least about two weeks or four weeks.

30. The article of manufacture of claim 29, wherein the package insert includes instructions for administration of the PCSK9 antagonist antibody to an individual being treated with a statin.

31. The article of manufacture of claim 30, wherein the statin is atorvastatin, cerivastatin, fluvastatin, lovastatin, mevastatin, pitavastatin, pravastatin, rosuvastatin, simvastatin, or any pharmaceutically acceptable salts, or stereoisomers, thereof.

32. The article of manufacture of claim 29, wherein the instructions are for administration of an initial dose by intravenous or subcutaneous injection and at least one subsequent dose by intravenous or subcutaneous injection.

33. The article of manufacture of claim 29, wherein a plurality of subsequent doses are administered.

34. The article of manufacture of claim 29, further comprising a label on or associated with the container that indicates that the composition can be used for treating a condition characterized by an elevated low-density lipoprotein cholesterol level in the blood.

35. The article of manufacture of claim 34, wherein the label indicates that the composition can be used for the treatment of hypercholesterolemia, atherogenic dyslipidemia, hyperlipidemia, atherosclerosis, cardiovascular disease, or acute coronary syndrome (ACS).

36. The article of manufacture of claim 29, wherein the antibody is L1L3.
